(19) United States
(12) Patent Application Publication
(10) Pub. No.: US 2026/0345170 A1
(43) Pub. Date: Nov. 5, 2026

(54) MEDICAL DEVICES COMPRISING DETACHABLE BALLOON-LIKE IMPLANTS AND METHODS OF MANUFACTURING AND USE

(71) Applicant: Artio Medical, Inc., Prairie Village, KS (US)

(72) Inventors: F. Nicholas Franano, Prairie Village, KS (US); Zachary Nelson, Menlo Park, CA (US); Vera Shinsky, Menlo Park, CA (US); Blaise Baxter, Prairie Village, KS (US)

(21) Appl. No.: 19/120,209

(22) PCT Filed: Oct. 10, 2023
(86) PCT No.: PCT/US2023/076421

§ 371 Date: Apr. 10, 2025

Related U.S. Application Data
(60) Provisional application No. 63/378,965, filed on Oct. 10, 2022.

(51) Int. Cl. A61B 17/12; A61B 17/00; A61B 90/00

(52) U.S. Cl. CPC A61B 17/12113; A61B 17/00234; A61B 17/12036; A61B 17/12172; A61B 90/39; A61B 2017/00238; A61B 2017/00305; A61B 2017/00477; A61B 2017/00557; A61B 2017/00867; A61B 2017/12054; A61B 2090/3966

(57) ABSTRACT

The present disclosure relates to medical devices comprising detachable balloon-like implants and catheter assemblies, wherein the detachable balloon-like implants include polymer materials. Various means of attachment and detachment of the balloon-like implants to the catheter assemblies are described. Kits and uses of systems having one or more medical devices, detachable balloon-like implants, and elongated or expandable bodies are also disclosed.

### CROSS-REFERENCE TO RELATED APPLICATIONS

This application claims the benefit of U.S. Provisional Application No. 63/378,965, filed Oct. 10, 2022, which is hereby incorporated by reference, in its entirety and for all purposes.

### FIELD OF THE PRESENT DISCLOSURE

The present disclosure relates to a first medical device comprising a balloon-like implant and a catheter or catheter assembly, wherein the balloon-like implant can be delivered to a desired location in a human patient in a pleated and folded form, expanded, and detached from the catheter or catheter assembly in a manner that allows the expanded balloon-like implant to remain in the patient while the catheter or catheter assembly is removed from the patient, a “detachable balloon catheter”. The present disclosure also describes a second medical device comprising expandable bodies configured for use with detachable balloon catheters, wherein all or a portion of an expandable body is used to help maintain the size, shape, or position of the detached balloon-like implant of the detachable balloon catheter. Systems and kits comprising a detachable balloon catheter medical device and one or more expandable body medical devices are described. The use of detachable balloon catheter medical devices or systems comprising a detachable balloon catheter medical device and one or more expandable body medical devices to reduce the flow of blood or other biological fluids in saccular aneurysms, arteries, veins, left atrial appendages, paravalvular leaks, other blood-containing structures, biological conduits, and biological spaces is described. Kits comprising a detachable balloon catheter medical device and one or more expandable body medical devices, optionally along with other medical devices are also disclosed.

### BACKGROUND OF THE PRESENT DISCLOSURE

Endovascular medical devices for arterial occlusion also include self-expanding vascular plugs that can also be pushed through a catheter and deposited in the lumen of the target arterial segment. There are benefits to using vascular plugs for arterial occlusion. The devices can be placed with greater accuracy than coils. Also, a single device is often all that is needed for arterial occlusion, reducing the complexity of the treatment. However, as with coils, there are drawbacks. The devices are often stiff, making them difficult to place in small, distal, and tortuous arteries. Like coils, vascular plugs present a porous barrier to the flow of blood, which can increase the time required for the devices to completely occlude an artery. They also frequently show late recanalization of treated arterial segments.

Previous attempts have been made to develop and use detachable balloons for the occlusion of arteries, wherein polymer balloons are inflated to fill the lumen of the target arterial segment and detached from a catheter. Compliant, detachable polymer balloons joined to catheters are advanced into the target arterial segment, inflated, and detached in an attempt to completely fill and occlude the arterial segment. There are two major benefits to using detachable balloon catheters for arterial occlusion. First, these devices have a low profile and are very flexible, enabling treatment of small, distal, and tortuous arteries. Second, after inflation, the expanded, compliant balloons generally conform well to irregularities in the surrounding vessel wall and often provide a good seal against the artery wall and good acute occlusion performance. However, these devices and their associated methods of use have several drawbacks and have been mostly abandoned in favor of other devices and methods. First, the devices are typically made of compliant polymers such as latex and silicone that generally resist tissue incorporation. This reduced fixation of the devices to the artery wall increases the risk of balloon migration. Second, the balloons are elastic and use valves to preserve a high internal pressure after detachment that is needed to maintain their expanded size and shape. Unfortunately, there is a substantial rate of valve failure resulting in balloon deflation, often leading to recanalization of the treated arterial segment and increasing the risk of balloon migration. Third, the polymers used to fabricate the balloons are biodegradable in vivo. The breakdown of the wall of the balloons increases the risk of balloon collapse and recanalization of the treated arterial segment.

There remains an unmet clinical need for medical devices, catheter-based medical devices, systems, and methods for effectively and reliably occluding, embolizing, sealing, or reducing the flow of blood in arteries and arterial segments.

Devices are needed which are low profile and highly flexible, easy to use, and can be quickly placed with a high degree of precision. Additionally, devices are needed which result in immediate and complete arterial occlusion with just one or a few devices that have a reasonable cost and require a limited number of sizes and shapes to occlude most arteries. Finally, devices are needed which offer durable and permanent occlusion of arteries with low rates of device collapse, compression, compaction, and migration, and low rates of recanalization of the treated arterial segment.

The left atrial appendage (LAA) is a small, saccular protrusion of the muscular wall of the left atrium. The LAA is generally regarded as a vestigial structure, with no clear function. In normal hearts, the heart contracts with each heartbeat. During left atrial contraction, blood in the left atrium and LAA is expelled into the left ventricle. Electrical impulses control the timing of the beating of various chambers of the heart.

When these impulses do not travel in an orderly fashion, fast and chaotic impulses can occur, reducing the coordination of atrial contraction and limiting the expulsion of blood from the left atrium and LAA, a condition known as atrial fibrillation which affects an estimated 2.7 million Americans. LAA is a source of clots that can travel to the brain and cause strokes. Atrial fibrillation (AF) exacerbates the occurrence of stroke, and for patients with AF, there is a benefit to occluding the LAA to prevent strokes.

There remains an unmet clinical need for medical devices, catheter-based medical devices, systems, and methods for effectively and reliably occluding, embolizing, sealing, or reducing the flow of blood in the LAA. Devices are needed which are low profile and highly flexible, easy to use, and can be quickly placed with a high degree of precision. Additionally, devices are needed which offer immediate and complete occlusion of the LAA, present a solid, well washed surface to the left atrium that endothelializes quickly, and result in high rates of durable and permanent occlusion of the LAA.

There remains an unmet clinical need for medical devices, catheter-based medical devices, systems, and methods for effectively and reliably occluding, embolizing, sealing, and reducing the flow of blood in veins and venous segments. Devices are needed which are low profile and highly flexible, easy to use, and can be quickly placed with a high degree of precision. Additionally, devices are needed which result in immediate and complete vein occlusion with just one or a few devices that have a reasonable cost, and require a limited number of sizes and shapes to occlude most veins. Finally, devices are needed which offer durable and permanent occlusion of veins with low rates of device collapse, compression, compaction or migration, and low rates of recanalization of the treated vein segment.

There remains an unmet clinical need for medical devices, catheter-based medical devices, systems, and methods for effectively and reliably occluding paravalvular leak paths. Devices are needed which are low profile and highly flexible, easy to use, and can be quickly placed with a high degree of precision. Additionally, devices are needed which result in immediate and complete occlusion of the leak path, and have surfaces that are resistant to thrombus formation, and endothelialize quickly. Finally, devices are needed which offer durable and permanent occlusion of the leak path with low rates of device collapse, compression, or compaction, and recanalization.

There remains an unmet clinical need for medical devices, catheter-based medical devices, systems, and methods for effectively and reliably occluding biological conduits. Devices are needed which are low profile and highly flexible, easy to use, and can be quickly placed with a high degree of precision. Additionally, devices are needed which result in immediate and complete conduit occlusion with just one or a few devices that have a reasonable cost and require a limited number of sizes and shapes to occlude most conduits. Finally, devices are needed which offer durable and permanent occlusion of conduits with low rates of device collapse, compression, compaction or migration, and low rates of recanalization of the treated conduit segment.

### SUMMARY

The present disclosure generally relates to expandable medical devices for placement in a biological space. In one aspect, the medical device includes a compressed balloon-like implant configured for implantation in the biological space. The balloon-like implant includes a distal region, a proximal region generally opposite the distal region, an intermediate region transitioning from the distal region to the proximal region, a first axis extending proximal-distal between the proximal region and distal region, and a second axis perpendicular to the first axis. The balloon-like implant also includes a wall extending generally continuously from the proximal region through the intermediate region, to the distal region, with an exterior surface and an interior surface. The interior surface defining a central void or interior volume, wherein the central void or interior volume is configured to be modified based on deployment.

The balloon-like implant also includes an opening in the wall at a proximal end of the proximal region that allows for the passage of fluid from a first catheter into the central void or interior volume and also allows for passage of a portion of a second catheter into the central void or interior volume. An opening in the wall at a distal end of the distal region allows for the passage of a portion of the second catheter out of the central void or interior volume of the balloon-like implant.

According to one aspect, the first catheter which, along with the second catheter, defines a first lumen to allow for passage of fluid from a proximal end of the first catheter to a distal end of the first catheter, and into the central void or interior volume of the balloon-like implant. The first catheter can include a proximal end that is coupled to a first proximal hub and a distal portion that is operably coupled or joined to the opening in the wall of the proximal region of the balloon-like implant. The distal portion includes a port configured to accept a guidewire.

According to one aspect, the second catheter defines a second lumen configured to accept at least one of the guidewire and an expandable device or combinations thereof. The second catheter includes a proximal end that is coupled to a proximal hub, a proximal portion that passes through the proximal hub of the first catheter, a distal portion that passes through the proximal opening of the balloon-like implant, a distal portion that passes through the central void or interior volume of the balloon-like implant, a distal portion that engages or passes through the distal opening in the balloon-like implant, and a distal end that is open.

According to yet another aspect, the expandable device anchors and supports the balloon-like implant after being deployed. The expandable device includes a first portion located at the distal end of expandable device. the first portion is configured to anchor the balloon-like implant when fully deployed. A second portion is located at the proximal end of the expandable device. The second portion is configured to expand within the balloon-like implant when fully deployed thereby providing support to the balloon-like implant. A waist is located between the first portion and second portion and the waist is configured to engage with the opening in the wall of the distal region when fully deployed.

According to one aspect, the second catheter can be moved forward or backward while the balloon-like implant remains fixed in position. Further all, or a portion of, the expandable device can be placed through the lumen of the second catheter into a biological space wherein the first portion of the expandable device is adjacent to the balloon-like implant, passing through the second catheter until the waist is exposed into the biological space. After the waist is exposed, the balloon-like implant or the first portion of the expandable device is positioned with the first or second catheter, respectively, until the waist of the expandable device is adjacent yet distal to the distal opening of the balloon-like implant and the second catheter is adjacent yet distal to the distal opening of the balloon-like implant. The second catheter can be pulled back proximally until a distal tip of the second catheter is located in the central void or interior volume of the balloon-like implant, and a second portion of the expandable device can be deployed through the lumen of the second catheter and placed into the central void or interior volume of the balloon-like implant.

According to one aspect, the passage of fluid through the first catheter into the central void or interior volume of the balloon-like implant can result in expansion of the balloon-like implant. After expansion of the balloon-like implant and placement of the second portion of the expandable device, the second catheter can be withdrawn proximally and the first catheter can be separated from the expanded balloon-like implant and removed from the patient while the balloon-like implant and the first and second portions of the expandable device remain in the patient.

The present disclosure also relates to an expandable braid device configured for permanent implantation in a biological space. The expandable braid device includes a distal portion having a first distal diameter, a proximal portion having a first proximal diameter and is generally opposite the distal portion. The braid device also includes a waist transitioning from the distal portion to the proximal portion, a first axis extending proximal-distal between the proximal portion and distal portion, and a second axis perpendicular to the first axis.

The braid device further includes an attachment coupler located at a proximal end of the proximal portion. The attachment coupler configured to releasably engage the expandable braid device to a delivery structure. The braid device also includes a plurality of wires extending generally continuously from the proximal portion to the distal portion. The plurality of wires including a first pitch region having a first pitch. The present disclosure also relates to methods of using the expandable device, methods of using the expandable braid device, as well as kits including the expandable device and/or the expandable braid along with instructions for use.

### BRIEF DESCRIPTION OF FIGURES

FIGS. 1A-B are planar views of one embodiment of a balloon-like implant having both proximal and distal necks with its overall geometric dimensions defined.

FIG. 2 is a planar view of one embodiment of a polymer balloon-like implant having both proximal and distal necks and detachment buffer zone with its overall geometric dimensions defined.

FIG. 3 is a planar view of one embodiment of an expandable body with both proximal and distal portions with its overall geometric dimensions defined.

FIGS. 4A-E provide various examples of expandable bodies where portions of the expandable bodies have varying diameters and pitch.

FIGS. 5A-B provides views of an expandable body with twisted wires and waist.

FIG. 5C shows an example of a valve seal around the high-density braided waist.

FIG. 6 provides a planar view of a portion of the distal end of the medical devices including a port for a dual delivery system.

FIGS. 7A-H are partial cross-sectional views of balloon-like implants incorporating telescoping structures within their necks according to eight embodiments.

FIGS. 8A-D are planar views showing one embodiment of a detachable balloon-like implant delivery system comprising a guidewire and three catheters, including details of the proximal configuration and purpose of each catheter.

FIGS. 9A-C are planar and cross-sectional views showing proximal hub configurations of one embodiment of a detachable balloon-like implant delivery system before and after unlocking & retracting the second catheter from the first catheter.

FIGS. 10A-C are planar and cross-sectional views showing proximal hub configurations of one embodiment of a detachable balloon-like delivery system before and after unlocking & retracting the second catheter from the first catheter and the first catheter from the third catheter.

FIGS. 11A-E are cross-sectional detail views showing a first sequence of operation of a mechanical latch attachment system according to one embodiment.

FIGS. 12A-F are schematics showing the occlusion of arteries and veins using a detachable balloon catheter with adjunctive placement of vascular supports within the expanded balloon-like implant and an expandable retention structure deployed during treatment to secure the balloon-like implant.

FIGS. 13A-B shows example shapes of braid component dimensions and configurations according to some embodiments.

FIG. 14A-C is a photograph of an expandable body that includes a radiopaque core wire according to one embodiment.

### DETAILED DESCRIPTION

The present disclosure generally relates to medical devices 1 which can be used alone or in combination to treat human patients. When describing these medical devices, the proximal end generally refers to the end that is closest to the physician. The distal end generally refers to the end that is pushed or advanced into the patient. For individual components of medical devices described herein, this same proximal and distal orientation is generally maintained. In reference to the detachable balloon-like implant 10 portion of medical devices described herein, a first axis 706 extends along the centerline of the device between the proximal region 110 and the distal region 120 of the detachable balloon-like implant 10, and a second axis 708 extends perpendicular to the first axis 706.

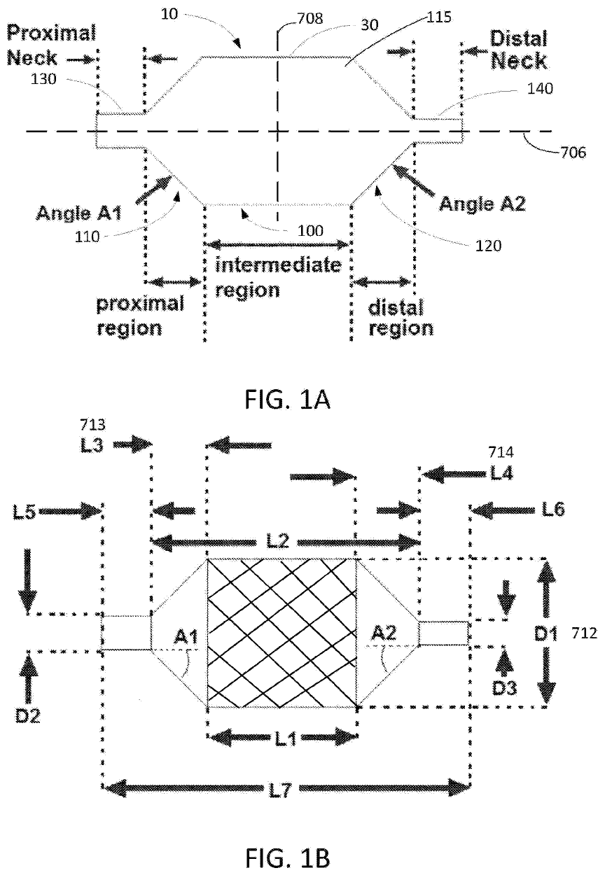

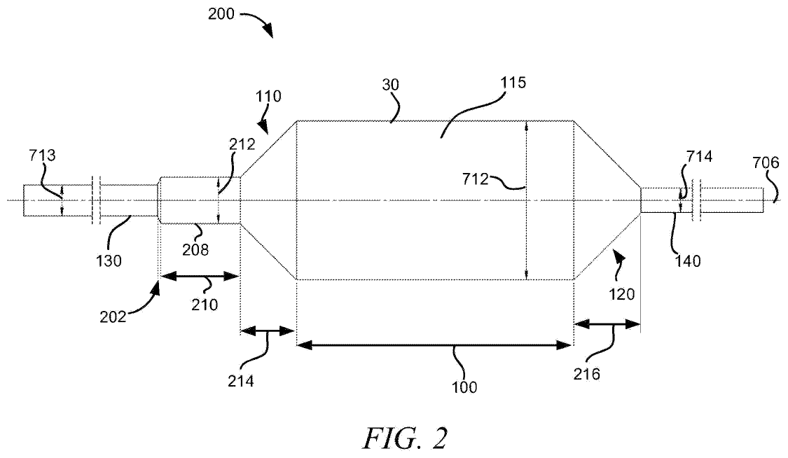

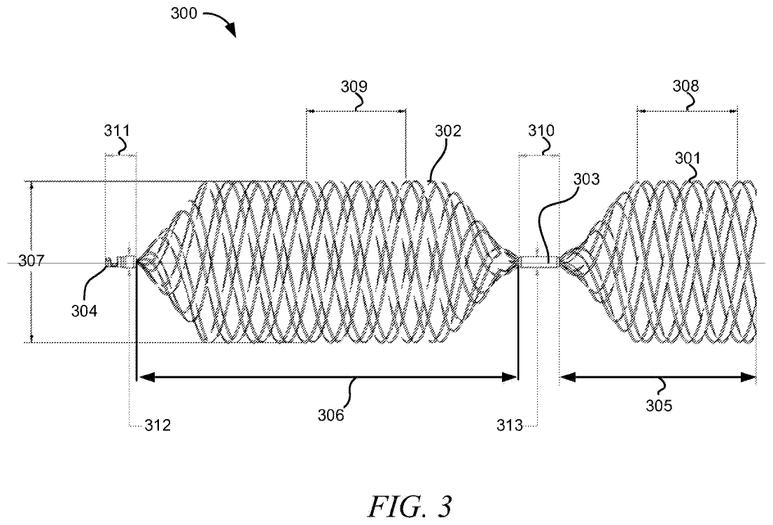

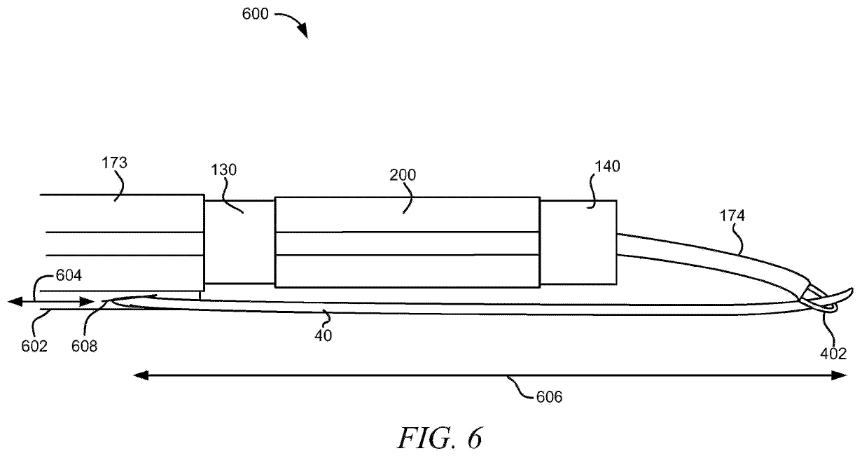

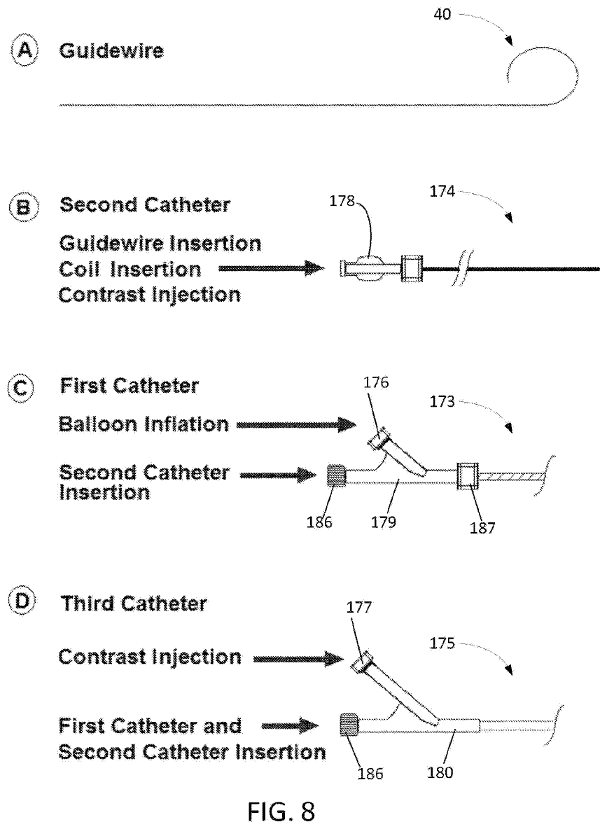

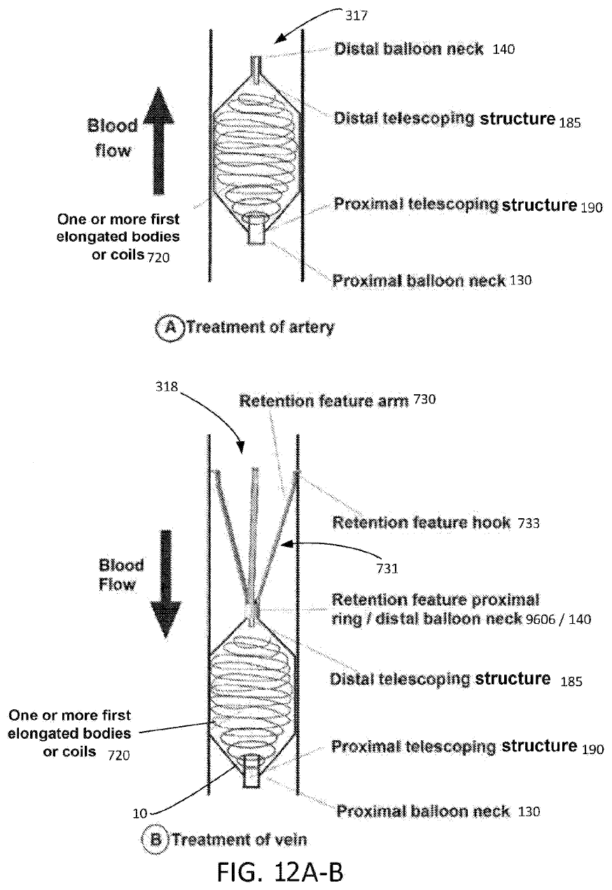

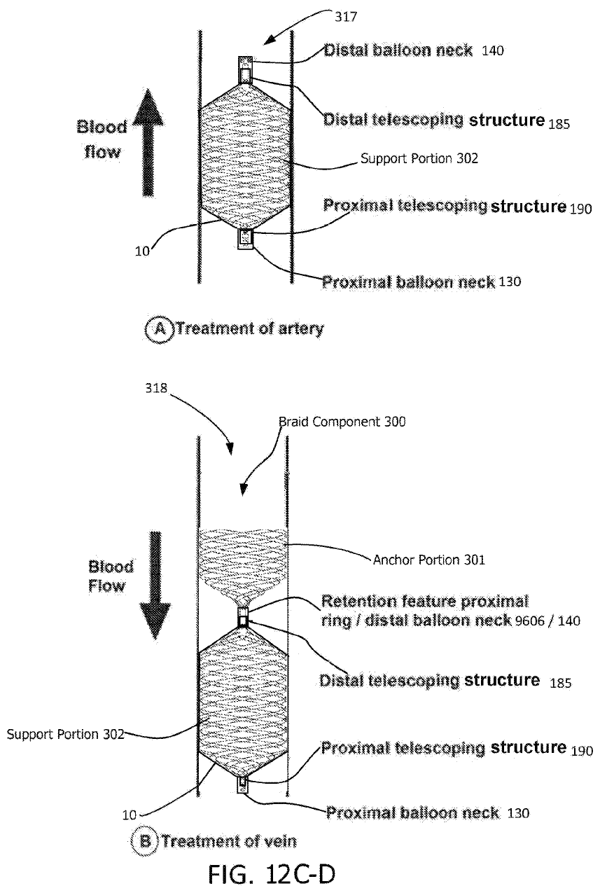

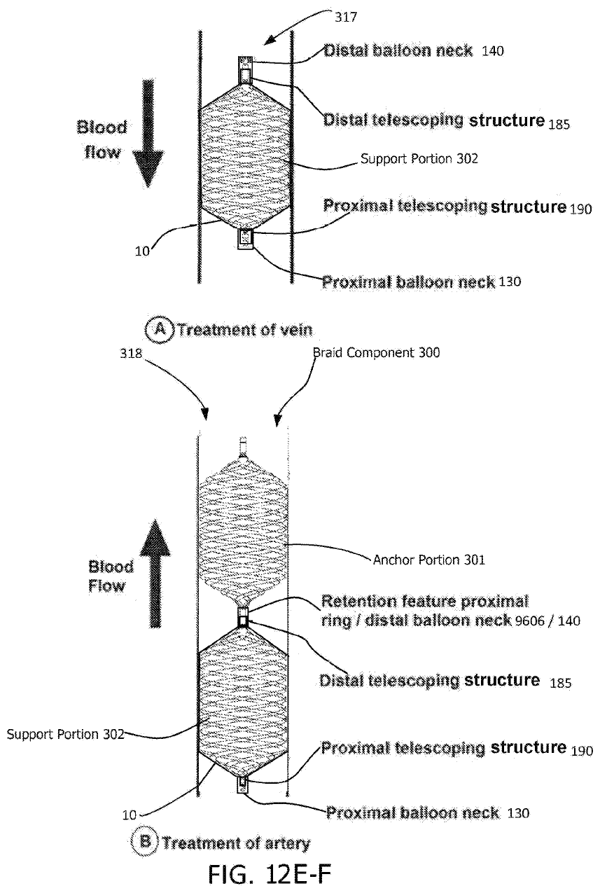

The present disclosure relates to medical devices 1 that comprise a detachable balloon-like implant 10, a wire for interacting with the balloon-like implant and/or an expandable device, and a catheter or catheter assembly 5, wherein the detachable balloon-like implant 10 is configured for expansion with fluid and detachment from the catheter or catheter assembly 5 in vivo. After separation of an expanded detachable balloon-like implant 10 from a catheter or catheter assembly 5, the detachable balloon-like implant 10 is configured to maintain an expanded configuration. Herein, these devices are also called “detachable balloon catheters” or “first medical devices” 1. In some examples, the detachable balloon-like implant can be reattached and moved within or removed from the patient. The term balloon-like implant as used herein refers to a hollow structure with a nonporous wall 30 comprising a light or thin material that can be inflated or expanded, including with the injection of a fluid, gas, liquid, combination of the same, etc., into a central void 115, as shown in FIGS. 1A and 2. As used herein, a balloon-like implant can also be called a “hollow expandable structure” or “expandable hollow structure.” Various shapes, lengths, widths, and sizes of detachable balloon-like implants 10 that can be compressed and then expanded in a permanent or semi-permanent configuration are described. In some embodiments, any shape that can fill a void, remain or be supported to remain expanded, and include various widths and lengths can be utilized. Balloon-like implants with various retention structures 731, as shown in FIG. 12B, and surface textures and that reduce the risk of detachable balloon-like implant 10 migration in vivo are described. Detachable non-porous, pleated, and folded, flexible polymer balloon-like implants, such as balloon-like implant 200 shown in FIG. 2, having a braid component 300, shown in FIG. 3, to anchor the balloon-like implant in place are described, as shown in FIGS. 2-3, 4A-E, and 5A-B, along with a dual delivery system 600, shown in FIG. 6, with a mechanical detachment system. Various catheters and catheter assemblies 5 for in vivo use are described, including catheters 5 configured for detachable balloon-like implant 10 inflation, catheters 5 configured to accept a guidewire 40, catheters 5 configured for delivery of expandable or elongated bodies 720, catheters 5 configured for injection of radiographic contrast, and catheters 5 configured to constrain retention structures 731 prior to placement as shown in FIGS. 8A-D, 9A-C, and 10A-C. In some embodiments, the retention structure 731 can be a distal anchor portion 301 of braid component 300. It should be noted that guidewire 40 can be a catheter, such as a microcatheter.

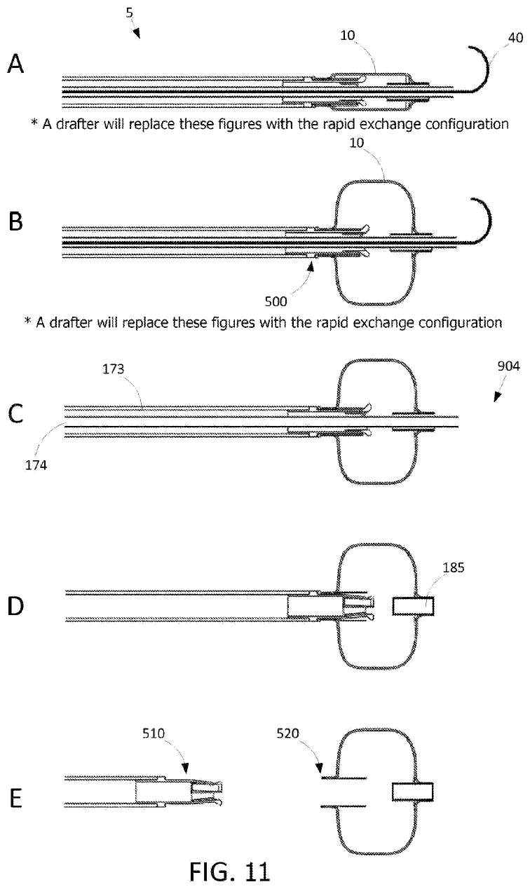

Various means of attaching detachable balloon-like implants 10 to catheters and catheter assemblies 5 are described, including a mating or coupling of parts. A friction fit can be made using an elastomeric or resilient tubular structure. A friction fit can be made using an elastomeric or resilient valve. Glues and adhesives and other bonding methods can be used in various possible embodiments. Various means of detaching balloon-like implants 10 from catheters and catheter assemblies 5 are described, including a decoupling of mated parts, as shown in FIGS. 11A-E, electrolysis, and applying heat or electricity to erode/melt specified areas of coupled parts, combinations of the same, etc., in various possible examples. In some embodiments, a pulling of a catheter or catheter assembly 5 away from an expanded detachable balloon-like implant 10 by overcoming a friction fit can be used to detach balloon-like implants 10 from catheters and catheter assemblies 5.

Various configurations of detachable balloon-like implants 10 and detachable balloon catheters 1 are described, along with the associated methods of manufacturing them. In one example, the detachable balloon-like implant 10 portion of a detachable balloon catheter 1 is configured in a compressed, collapsed, or pleated and folded form and configured for permanent implantation in a human patient. As shown in FIG. 1A, the detachable balloon-like implant 10 comprises a distal region 120, a proximal region 110 generally opposite the distal region 120, and an intermediate region 100 transitioning between the proximal and distal regions 110 & 120. A first axis 706 extends along the centerline of the device between the proximal region 110 and the distal region 120. A second axis 708 extends perpendicular to the first axis 706. A wall 30 extends generally continuously from the proximal region 110, through the intermediate region 100, to the distal region 120. The wall 30 has an exterior surface and an interior surface, the interior surface defining a central void 115 or interior volume. The detachable balloon-like implant 10 can have an opening in the wall 30 at the proximal region 110 that allows for the passage of fluid from a first catheter 173 into the central void 115 or interior volume of the balloon-like implant 10 and also allows for passage of a portion of a second catheter 174 into the central void 115 or interior volume of the balloon-like implant 10. The detachable balloon-like implant 10 also can have an opening in the wall 30 of the distal region 120 that allows for the passage of a portion of the second catheter 174 out of the central void 115 or interior volume of the balloon-like implant 10. In some examples, the angles of the proximal 110 and distal 120 region can be about 45 degrees for balloon-like implant folding, balloon-like implant expansion, and balloon-like implant compliance where compliance is a measure of outer diameter shrinkage caused by polymer chain slippage of the balloon-like implant structure which can be due to internal stresses and where the slippage is a function of time and heat. It should be noted that the angles of the proximal 110 and distal 120 region can range from 30 to 60 degrees or can be any suitable angle, or combination thereof in various possible examples.

Various configurations of catheter assemblies are described. As shown in FIGS. 8B-C and 9A-C, the first catheter 173, along with the second catheter 174, defines a first lumen 162 of annular cross-section to allow passage of fluid from a proximal end of the first catheter 173 to a distal end of the first catheter 173, and into the central void 115 or interior volume of the detachable balloon-like implant 10. The first catheter 173 further comprises a proximal end that is coupled to a first proximal hub 179, and a distal portion that is operably coupled or joined to the opening in the wall 30 of the proximal region 110 of the balloon-like implant 10. The second catheter 174 defines a second lumen 163 of circular cross-section configured to accept at least one of a guidewire 40, an elongated or expandable body 720, or a solidifying fluid. The second catheter 174 comprises a proximal end that is coupled to a second proximal hub 178; a proximal portion that passes through the proximal hub 179 of the first catheter 173; a distal portion that passes through the proximal opening, central void 115, and distal opening of the balloon-like implant 10; and a distal end that is open. The passage of fluid through the first catheter 173 into the central void 115 or interior volume of the balloon-like implant 10 can result in expansion of the balloon-like implant 10.

In one embodiment, a wire including caps made of non-porous materials (e.g., polymer materials or metal), the caps positioned at opposing ends, can be deployed in the biological space to resist or block the flow fluids in the biological space and to prevent the wires from piercing or otherwise damaging the biological space. The wire or plurality of wires with caps at opposing ends can be placed inside a balloon-like implant. In some embodiments, the wire or plurality of wires can be placed outside of the balloon-like implant thereby creating a scaffolding to support the balloon-like implant within the biological space and protecting the balloon-like implant from being compressed. In some embodiments, the wire can be a plurality of wires brought together and held by caps at opposing ends, the resulting structure utilized to prevent the passage of fluids in the biological space. In yet another embodiment, the blockage of fluid through the biological space can be accomplished with the deployed balloon-like implant, the deployed balloon-like implant including caps made of non-porous materials at opposing ends to resist or block the flow of fluids through the balloon-like implant.

The present disclosure also relates to medical devices that comprise an elongated or expandable body 720. Herein, these devices can also be called “second medical devices”. As used herein, an elongated body 720 is a long, thin, flexible structure that can be pushed or carried through the lumen of a catheter and implanted in a patient. Elongated bodies 720 can occupy space and form complex shapes, but do not expand during or after placement. As used herein, an expandable body 720 is a long, thin, flexible structure that can be pushed or carried through the lumen of a catheter in a constrained, collapsed, compressed, or pleated and folded form and implanted in a patient, wherein at least portions of the expandable body 720 can expand in size during or after placement. In one embodiment, the expandable body 720 is a solid body. In another embodiment, the expandable body 720 is a solid member with a wire running through the expandable body 720. Elongated and expandable bodies 720 that can be used with a first medical device comprising a detachable balloon catheter are described.

According to various embodiments, the expandable member 720 can be a braid component 300, such as a nitinol braid, that can be deployed first (i.e., before inflating the balloon-like implant) and expanded in the vessel. In some embodiments, the braid component 300 can be one portion. In various other embodiments, the braid component 300 can be two or more portions. In some embodiments, the braid component can include an anchor portion and a support portion. In this regard, the anchor portion is deployed or positioned first, and the support portion can then be deployed inside the balloon-like implant, thereby anchoring the braid and balloon-like implant in the vessel and providing support to the balloon-like implant.

In some embodiments, the braid component can include radiopaque wires that are visible on x-ray to aid in visualizing the portion of the braid component disposed outside of the balloon-like implant (e.g., the anchoring portion) and can include platinum or other radiopaque material on the portion inside of the balloon-like implant (e.g., the support portion). In this regard, using radiopaque materials can allow the anchor and the compaction of the stent or balloon-like implant itself to be visible thus making easier to know its position. The radiopaque wires can also aid in visualizing the braid component within the balloon-like implant to ensure sufficient support of the balloon-like structure and that the braid component has been properly deployed inside the balloon-like implant.

Expandable bodies, such as braid component 300, can be made from wire, polymer, other flexible materials, solid materials, and combinations of the same in various possible examples. In some embodiments, the expandable bodies can include 6 to 48 wires. In one embodiment, the expandable body includes 12 wires. In some embodiments, the wires can have a size or diameter in a range from 0.05 mm to 0.3 mm. In one embodiment, the wire sizes or diameter can be in a range from 0.05 mm and 0.08 mm. In another embodiment, the wire sizes or diameter can be in a range from 0.08 mm and 0.15 mm. In yet another embodiment, the wire sizes or diameter can be in a range from 0.15 mm and 0.18 mm. In some embodiments, the expandable body can be a solid body including non-flexible materials. Expandable bodies are generally formed from self-expanding materials or generally formed in a manner that renders the expandable body self-expanding. Examples of expandable bodies include self-expanding wires, nitinol wires, assemblies of wires, assemblies of metal wires, assemblies of metallic wires, assemblies of polymer strands, assemblies of wires or strands comprising metal and polymer, assemblies of coiled wires, assemblies of coiled metal wires, assemblies of coiled metallic wires, assemblies of coiled polymer strands, assemblies of coiled structures comprising metal and wire, assemblies of strands, assemblies of polymer strands, assemblies of metal strands, assemblies of metallic strands, and assemblies of strands comprising polymer and metal, assemblies of braided wires, assemblies of braided metal wires, assemblies of braided metallic wires, assemblies of braided wires comprising metal and polymer, assemblies of braided strands, assemblies of braided polymer strands, assemblies of braided strands comprising polymer and metal, assemblies of woven wires, assemblies of woven metal wires, assemblies of woven metallic wires, assemblies of woven wires comprising metal and polymer, assemblies of woven strands, assemblies of woven polymer strands, assemblies of woven strands comprising polymer and metal, balloon-like implants, and combinations thereof.

In some embodiments, a balloon-like implant is mounted distally with a mechanical latch onto a single lumen, such as a first catheter, over a second catheter (e.g., a delivery catheter, over a second catheter). Upon reaching the desired location in the vascular system, the expandable body, such as a braid component, is deployed into the vessel. The balloon-like implant position can be adjusted, and the implant is then expanded, such as by a saline and/or contrast injection, or by expanding the expandable body, or a portion thereof, inside of the balloon-like implant. In some embodiments, the balloon-like implant can be supported by any support member, such as a conical or cylindrical support member placed inside the balloon-like implant. In one embodiment, the proximal support portion of the braid component is deployed inside of the balloon-like implant to anchor and support the balloon-like implant. Once deployed, a second catheter is retracted, and the delivery catheter is detached from the non-compliant balloon-like implant.

In some embodiments, a 3-24 mm diameter non-compliant balloon-like implant is mounted distally with a mechanical latch onto a single lumen, such as a delivery catheter, over a second catheter such as a microcatheter. In some embodiments, a <1-24 mm diameter non-compliant balloon-like implant is mounted distally with a mechanical latch onto a single lumen. Upon reaching the desired location in the vascular system, the distal anchoring portion of the braid component, such as a nitinol braid component, is deployed into the vessel. The balloon-like implant position can be adjusted, and the implant is then expanded, such as by a saline and/or contrast injection or by manipulation of a support member, for example by expanding an expandable body. The proximal support portion of the braid component is deployed inside of the balloon-like implant to anchor and support the balloon-like implant. In this regard, the distal portion of the expandable body can be positioned in the biological space to come in contact with the biological space, pressing against the biological space to secure itself and/or the balloon-like implant. A proximal support portion can then be expanded and/or positioned inside of the balloon-like implant. The proximal portion can expand both itself and the balloon-like implant thereby supporting the balloon-like implant by contacting the inner wall, or a portion thereof. In some embodiments, the balloon-like implant is expanded before the proximal support portion of the expandable body. Once deployed, a second catheter (e.g., a microcatheter) is retracted, and the delivery catheter is detached from the balloon-like implant and/or the expandable body.

In some embodiments, a solidifying fluid comprises an adhesive that can be injected as a fluid through the first lumen 162 or the second lumen 163 and into the central void 115 of the balloon-like implant 10 or injected into a biological space 904 adjacent to an expanded balloon-like implant 10, wherein the solidifying fluid becomes a solid or semi-solid after passing through the first lumen 162 or the second lumen 163. Some examples of solidifying fluids include adhesives such as cyanoacrylates or UV curable adhesives, ethylene vinyl alcohol, Onyx® copolymer, or particle that increases in viscosity at physiologic salinity. In some embodiments, the solid solidifying agent acts to help the expanded, detached balloon-like implant 10 of the detachable balloon catheter 1 resist collapse, compression, or compaction. In some embodiments, the solid solidifying agent acts to help maintain the position of the expanded, detached balloon-like implant 10 of the detachable balloon catheter 1. In some embodiments, the solid solidifying agent acts to reduce the flow of blood or other biological fluids or suspensions through treated arteries 317, veins 318, or other biological conduits. In some embodiments, the solid solidifying agent acts to occupy a biological space.

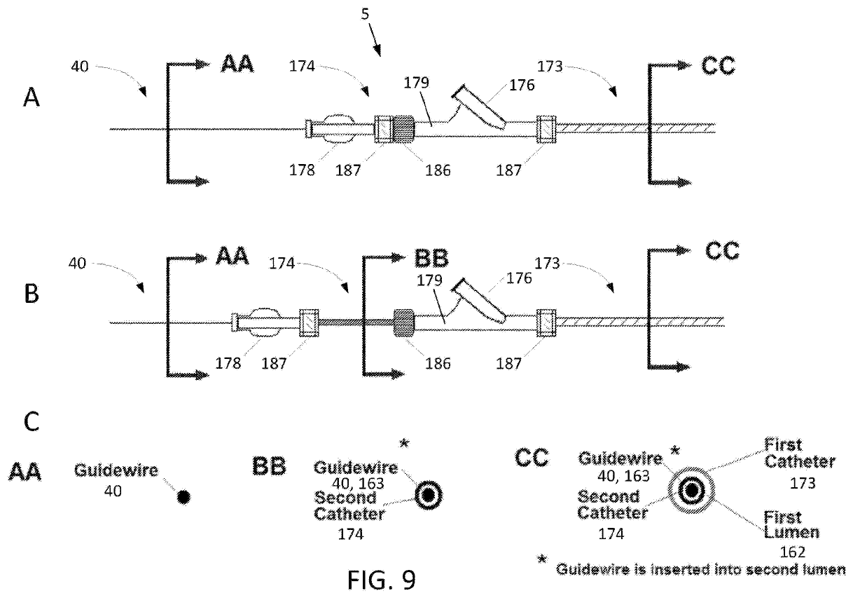

Continuing the deployment sequence following expansion of the detachable balloon-like implant 10, the second catheter 174 can be moved forward or backward while the balloon-like implant 10 remains fixed in position, as shown in FIGS. 9A-B. After removal of the guidewire 40, all or a portion of one or more second medical devices comprising an elongated or expandable body 720 or solidifying fluid can be placed through the lumen 163 of the second catheter 174 into a biological space adjacent to the balloon-like implant 10. In some embodiments, all or a portion of one or more second medical devices comprising an elongated or expandable body 720 or solidifying fluid can be placed through the lumen or prepositioned in the lumen at the distal end of the second catheter. The second catheter 174 can then be pulled back until the distal tip of the second catheter 174 is located in the central void 115 of the balloon-like implant, while the first catheter 173 and the balloon-like implant 10 remain fixed in position. All or a portion of one or more second medical devices comprising an elongated or expandable body 720, solidifying fluid, or other balloon-like implant support material can be passed through the second lumen 163 of the second catheter 174 and placed into the central void 115 of the balloon-like implant 10. In some embodiments, the second medical device comprising an elongated or expandable body, solidifying fluid, or other balloon-like implant support material can be passed through the lumen of the first catheter 173 and deployed before or in conjunction with the balloon-like implant. In some embodiments, the second medical device can be guided and/or positioned by a guidewire running through a dual delivery system 600, shown in FIG. 6. The dual delivery system can include a side catheter 602 with an opening, also referred to herein as a “rapid exchange port”, with opening 608 shown in FIG. 6 and described below. The guidewire can pass through a lumen of the side catheter and into and through the opening 608 thereby exposing a portion of the guidewire to an area outside of the delivery catheter. Once outside of the side catheter, the guidewire can interact with a distal loop of the second medical device by engaging the distal loop to guide the first and second medical device over a guidewire path. In this regard, the dual delivery system can allow the first medical device, such as polymer balloon-like implant 200, and a second medical device or expandable body, such as braid component 300 shown in FIG. 3, to be delivered and positioned to an area inside the body together at the same time. In some embodiments, the dual delivery system can allow the second medical device or expandable body, such as braid component 300, to be delivered and/or positioned before the first medical device, such as flexible polymer balloon-like implant 200. It should be noted that the expandable body, or portion thereof, can be deployed or expanded before the balloon-like implant is expanded.

A medical device, such as braid component 300 shown in FIG. 3, which can include a distal anchor portion and a proximal support portion, can be advanced through the first catheter and guided into the aneurysm or vessel lumen by guidewire before the detachable balloon-like implant is deployed. Although a specific type of medical condition (e.g., aneurysm) is identified, it should be noted that the systems and methods consistent with this disclosure can be used to treat other conditions and can involve other areas of a body such as, saccular aneurysms, arteries, veins, left atrial appendages, paravalvular leaks, other blood-containing structures, biological conduits, and biological spaces, etc., and combinations of the same in various possible embodiments. In some embodiments, the guidewire runs through a portion of a side catheter located adjacent to the first catheter 173, such as rapid exchange port 608 shown in FIG. 6. In this regard, the braided medical device and balloon-like implant can be guided by the guidewire whereby a distal portion of guidewire does not pass through the implant and instead runs through the side catheter. Once the distal anchor portion of the braided medical device is positioned, the balloon-like implant can be inflated and positioned. The support portion of the braided medical device can then be deployed within the balloon-like implant thereby securing the balloon-like implant in a desired location. In some embodiments, the proximal support portion of the braided medical device can be deployed in conjunction with inflating the balloon-like implant.

In some embodiments, a compressed or collapsed balloon-like implant expandable body comprises a balloon-like implant 10 wherein portions of the wall 30 of the balloon-like implant 10 are squeezed or pressed together or into a much smaller space than the expanded balloon-like implant 10. In some embodiments, a constrained balloon-like implant 10 is forced and held into a smaller space or smaller diameter than the expanded balloon-like implant 10. In some embodiments, the balloon-like implant portion 10 of the detachable balloon catheter 1 is pleated, folded, or compressed into a shape that occupies a smaller space or smaller diameter than the expanded balloon-like implant 10, which is called a “deliverable configuration”. In some embodiments, the expandable body portion 720 of the second medical device is constrained or compressed into a shape that occupies a smaller space or smaller diameter than the expanded expandable body 720, which is also called a deliverable configuration.

Methods of treatment of saccular aneurysms, arteries 317, veins 318, left atrial appendages, paravalvular leaks, other blood containing structures, biological conduits, or other biological spaces using a detachable balloon catheter 1 with or without adjunctively using one or more elongated or expandable bodies 720 are also described.

### Balloon-Like Implants

A variety of detachable balloon-like implant shapes and sizes are described, as shown in FIGS. 1A, 1B, and 2. In some embodiments the detachable balloon-like implants 10 can be characterized to include a proximal region 110, an intermediate region 100, and a distal region 120, wherein the proximal and distal regions 110 & 120 are generally opposite each other. For each body, the proximal region 110, the intermediate region 100, and the distal region 120 form the unitary construction of the detachable balloon-like implant 10. For this characterization, the proximal region 110, the intermediate region 100, and the distal region 120 together form a “main body” of the detachable balloon-like implant 10, which excludes the proximal and distal necks 130 & 140. In some embodiments without an intermediate region 100 the detachable balloon-like implants can be characterized to include a proximal region 110 and a distal region, 120, wherein the proximal and distal regions 110 & 120 are generally opposite each other. For each of these bodies, the proximal region 110 and the distal region 120 form the unitary construction of the detachable balloon-like implant 10. For this characterization, the proximal and distal regions 110 & 120 together form a “main body” of the detachable balloon-like implant 10, which excludes the proximal and distal necks 130 & 140. The detachable balloon-like implants 10 can further be defined by a first axis 706 and a second axis 708 transverse to the first axis. 706. In one aspect, the first axis 706 extends between the proximal neck 130 and distal neck 140.

In some embodiments, detachable balloon-like implants 10, when expanded, are configured to assume a general shape comprising one lobe, excluding proximal and distal necks 130 & 140 or neck assemblies, if any. Some detachable balloon-like implants, when expanded, can be configured to assume a generally spherical, spheroid, oblate spheroid, prolate spheroid, ellipsoid, oblate ellipsoid, or a prolate ellipsoid shape, excluding proximal and distal necks 130 & 140 or neck assemblies, if any. Some detachable balloon-like implants, when expanded, comprise a proximal region 110, a distal region, 120, and an intermediate region 100. Other detachable balloon-like implants, when expanded, comprise a proximal region 110, a distal region 120, without an intermediate region 100. In some embodiments, the intermediate region 100 of a detachable balloon-like implant 10, when expanded, is generally cylindrical. In some embodiments, the detachable balloon-like implant 10, when expanded, is configured to assume a generally oblong or cylindrical shape, excluding proximal and distal necks 130 & 140 and neck assemblies, if any.

In some embodiments, as the braid component expands, it can need a length or space in order to open up. Without a dedicated area for this to occur, as the braid component expands, the microcatheter could kick back out of the balloon-like implant, proximal to the delivery catheter latch mechanism. The system can then detach, and with the system detached, an operator can lose the ability to keep pushing the braid component into the balloon-like implant potentially leaving the balloon-like implant inadequately filled and thereby compromised. In various embodiments, the proximal region or proximal neck can include a detachment buffer zone which can compensate for a kickback force (i.e., a force resulting from deployment of the implant support material) to prevent premature or inadvertent detachment of the balloon-like implant, such as balloon-like implant 10. The balloon-like implant support material, such as coils, braids, gels, etc., can cause the balloon-like implant to prematurely or inadvertently detach from the delivery catheter when the support material is deployed by pushing against the distal region of the balloon-like implant thereby separating the implant from the delivery catheter and/or mechanical latch system before it is intended to do so. The detachment buffer zone can include a length and a diameter within the proximal region or proximal neck of the balloon-like implant. In some embodiments, the detachment buffer zone length can range from <1-6 mm. In one embodiment, the detachment buffer zone length is 3 mm. The detachment buffer zone can be part of the balloon-like implant and therefore can be made of the same materials. The proximal end of the detachment zone can include a tapered aperture for receiving the proximal neck and/or delivery catheter. In this regard, the detachment buffer zone allows the balloon-like implant to compensate for the kickback force from deploying the support material thereby keeping the implant from separating prematurely or inadvertently.

In some embodiments, the detachable balloon-like implants 10 can be defined and described by the proximal region 110 and the distal region 120, where each region is generally a hemispheroid. The hemispheroid formed by each region and is further defined by a semi-major axis and semi-minor axis that can be parallel with the first axis 706 or the second axis 708, depending upon the lengths of each axis. In various embodiments, the hemispheroid of the proximal region 110 has a semi-major axis and semi-minor axis different from that of the distal region 120. In other embodiments, the hemispheroid of the proximal region 110 has a semi-major axis and semi-minor axis the same as that in the distal region 120. Similarly, for each distal and proximal region 110 and, respectively, the semi-major and semi-minor axis can differ from one another or be identical, so the corresponding region can have a generally shape of an oblate hemispheroid, a prolate hemispheroid, or a hemisphere. The detachable balloon-like implants 10 can also be fabricated in many other configurations that have generally spheroid or ellipsoid shapes.

For some detachable balloon-like implants, as shown in FIGS. 1A-B, wherein the intermediate region 100 is generally cylindrical and the proximal or distal regions 110 & 120 are conical, the proximal or distal regions 110 & 120 have a cone angle (defined as the angle between the wall 30 of the balloon-like implant and the first axis 706 of the balloon-like implant) of 20-75 degrees. In one embodiment, the shape of the intermediate region of the detachable balloon-like implants can be defined by the rotation, about the first axis 706, of a variable radius arc formed along the first axis 706, where the maximum radius for the variable arc is equal to either the maximum radius 711 of the distal region 720 or the maximum radius 710 of the proximal region 110, as measured along the second axis 708. For some embodiments, the expanded detachable balloon-like implant has a total length 709 along the first axis 706 that is less than or equal to the maximum diameter 712 of the expanded detachable balloon-like implant along the second axis 708.

As shown in FIGS. 1A-B, some detachable balloon-like implants 10, when expanded, are configured to have a maximum diameter of 2-48 mm when measured parallel to the second axis 708. Some detachable balloon-like implants 10, when expanded, are configured to have a maximum length of 2-80 mm when measured parallel to the first axis 706, excluding proximal and distal necks 130 & 140 and neck assemblies, if any. Some detachable balloon-like implants comprising a proximal region 110, intermediate region 100, and distal region 120, when expanded, are configured to have a maximum length of the main body or intermediate region 100 of 2-40 mm when measured parallel to the first axis 706, excluding the lengths of proximal and distal necks 130 & 140 and neck assemblies, if any. Some detachable balloon-like implants 10, when expanded, have a largest diameter as measured parallel to the second axis 708 that is greater than the largest length as measured parallel to the first axis 706, excluding the lengths of proximal and distal necks 130 & 140 and neck assemblies, if any. Some detachable balloon-like implants 10, when expanded, have a largest diameter as measured parallel to the second axis 708 that is equal to the largest length as measured parallel to the first axis 706, excluding the lengths of proximal and distal necks 130 & 140 and neck assemblies, if any. Some detachable balloon-like implants 10, when expanded, have a largest length, as measured parallel to the first axis 706, that is greater than the largest diameter as measured parallel to the second axis 708, excluding the lengths of proximal and distal necks 130 & 140 and neck assemblies, if any. In some embodiments, the expanded detachable balloon-like implants 10 have a length from the proximal neck 130 to the distal neck 140 of approximately 4-30 mm, or larger, and a maximum diameter 712 of approximately 2-30 mm, or larger. In some embodiments, the max length of the balloon-like implant can be approximately 54 mm including the cylindrical length of approximately 24 mm, each side's sloped length (approximately 2×12 mm=24 mm), and a buffer region length of approximately 6 mm. In some embodiments, the max length of the balloon-like implant can be approximately 78 mm including an approximately 48 mm cylindrical length, an approximately 24 mm combined side's sloped length, and an approximately 6 mm buffer region length. In some embodiments, the balloon-like implant can have a max length that can range between approximately 24 mm and 80 mm.

In one aspect, various configurations of the detachable balloon-like implants 10 can be obtained by independently varying the maximum length (also called “height”) along the first axis 706 of the proximal and distal regions 110 & 120, as shown in FIGS. 1A-B. For example, the height 713 of the proximal region 110 can be smaller than the height 714 of the distal region 120. In other examples, the height 713 of the proximal region 110 can be equal to the height 714 of the distal region 120. In other examples, the height 713 for the proximal region 110 can be larger than the height 714 for the distal region 120. While both detachable balloon-like implants 10 and have the same maximum diameter 712, the difference in the heights 713 & 714 of the proximal and distal regions 110 & 120, respectively, of each detachable balloon-like implant 10 results in different overall shapes for the detachable balloon-like implant 10.

In other embodiments, the heights 713 & 714 of the proximal and distal regions 110 & 120, respectively, can be varied independently to produce a wide variety of configurations of the detachable balloon-like implants 10. In a first embodiment, the height 713 of the proximal region 110 can be approximately 2 mm, while the height 714 of the distal region 120 is approximately 4 mm. In a second embodiment, the height 713 of the proximal region 110 can be approximately 3 mm, while the height 714 of the distal region 120 is also approximately 3 mm. In a third embodiment, the height 713 of the proximal region 110 can be approximately 2 mm, while the height 714 of the distal region 120 is approximately 3.5 mm. In a fourth embodiment, the height 713 of the proximal region 110 can be approximately 3 mm, while the height 714 of the distal region 120 is approximately 4 mm. It should be noted that the diameter of the balloon-like implant can range between approximately 2 mm to 30 mm, or larger, in various possible examples.

In some embodiments, as shown in FIG. 2, the medical device can include a balloon-like implant 200 having a proximal region 110 with a proximal region length 214 and distal region 120 with a distal region length 216. The balloon-like implant 200 can include an intermediate region 100, and proximal and distal necks 130 & 140, respectively. In one embodiment, the distal neck 140 includes a distal nosecone and/or a distal valve. The distal valve can allow a second catheter, such as catheter 174, and/or other components, such as braid component 300, to pass through and back out of the valve. The distal nosecone assembly, which can include a valve, can be attached to the distal neck 140 of the balloon-like implant 200 by an adhesive or can be attached by a mechanical attachment. The distal nosecone can include a lumen allowing a second medical device and/or catheter, such as catheter 174 and/or braid component 300, to pass through the distal nosecone. In an embodiment, the distal neck 140 is the distal nosecone. In an embodiment, the balloon-like implant 200 does not include a nosecone thereby allowing the guide catheter to be a smaller size. In this embodiment, a support, such as a hypotube, can plug the distal lumen of the balloon-like implant 200 aiding in deployment. In an embodiment, the lumen of the distal end of the balloon-like implant 200 can be left open. Balloon-like implant 200 can include, at a proximal end of the implant, a detachment buffer zone 208 where the detachment buffer zone 208 can compensate for a kickback force (i.e., a force resulting from deployment of the implant support material) to prevent premature or inadvertent detachment of the balloon-like implant 200. The detachment buffer zone 208 can include a buffer length 210 and a buffer diameter 212. The detachment buffer zone 208 can include a tapered proximal end 202 for receiving the proximal neck 130, second catheter 174, delivery catheter 173, and/or any suitable device or combinations thereof in various possible embodiments, within the proximal region 110 or proximal neck 130 of the balloon-like implant 200. The detachment buffer zone 208 allows the balloon-like implant 200, or a portion thereof, to provide a space where the support material can utilize during deployment without translating, by contact, the axial movement of the support material, such as a braid component 300, to the rest of the proximal region 110 or proximal neck 130 thereby possibly detaching the implant inadvertently or prematurely. The detachment buffer zone 208 allows the balloon-like implant 200, or a portion thereof, to compensate for the kickback force from deploying the support material thereby preventing the implant from separating prematurely or inadvertently.

In some embodiments, the balloon-like implant 200 can have a fully deployed length comprised of the distal region length 216, the intermediate region length 100, and the proximal region length 214. In some embodiments, the fully deployed length can also include the buffer zone length 210. In one embodiment, the fully deployed length can include the intermediate region length 100, the proximal region length 214, the distal region length 216, the buffer zone length 210, and the distal and proximal necks 130 and 140. It should be noted that the balloon-like implant can be comprised of any suitable length, which can include the distal and proximal regions, intermediate region, detachment buffer zone, proximal and distal necks, etc., or combinations thereof when fully deployed.

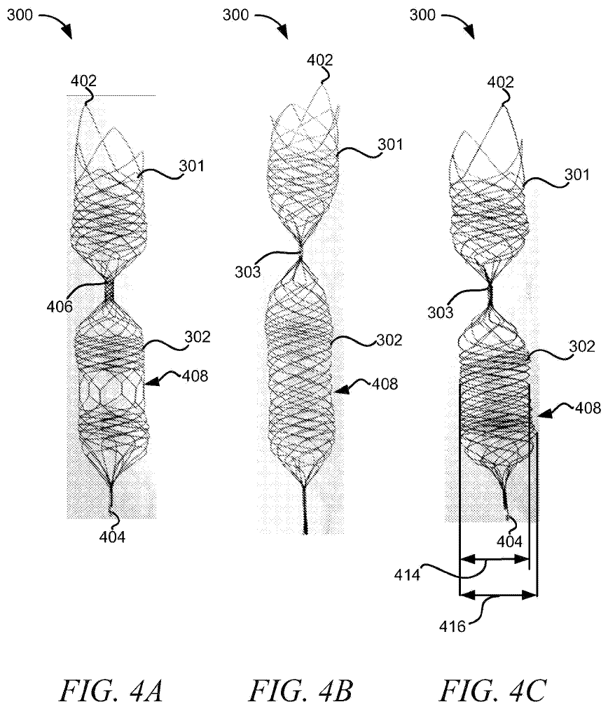

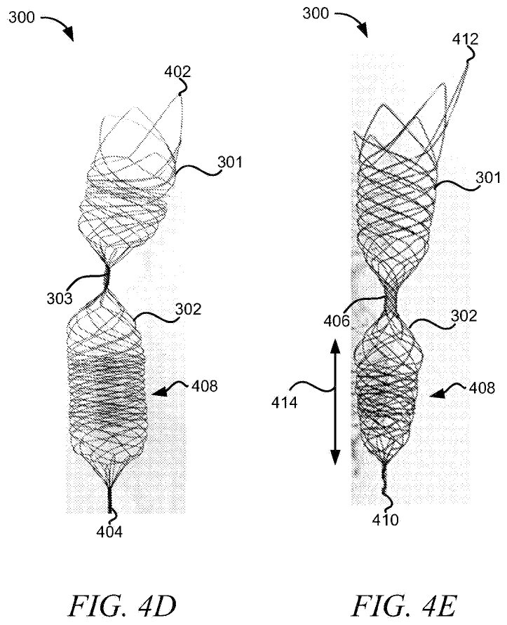

In some embodiments, as shown in FIG. 3, the medical device, such as expandable body 720, can be a braid component 300 having an anchoring portion 301 and a support portion 302. The anchoring portion 301 has a length 305 when fully deployed. The support portion 302 has a deployed length 306 which can be substantially (i.e., within a 10% margin) the same length as the fully deployed balloon-like implant 200. The support portion 302 includes fully deployed diameter 307 which can be substantially (i.e., within 10% margin) the same maximum diameter 712 as the fully deployed balloon-like implant 200. In this regard, the support portion 302 supports and/or anchors the fully deployed balloon-like implant 200 when deployed by filling the inside of the balloon-like implant 200. The support portion 302 can come in contact and support the nonporous wall 30 within the central void 115 and/or proximal and distal regions 110 and 120 of the balloon-like implant 200, shown in FIG. 2, when fully deployed. The anchoring portion 301 extends distally past the proximal support portion 302 with the distal 301 and proximal 302 portions separated by a waist which can include a tube, such as marker 303. The waist can incorporate a thicker wire, to aid visibility and increase strength during deployment, and/or can incorporate a high-density braid including twisted wires. The waist wire can be radiopaque and visible when imaged. The braid component 300 can include a mechanical detachment mechanism 304 located at the proximal end of the proximal support portion 302. The mechanical detachment mechanism has a length 311 and a diameter 312. The distal anchor portion 301 of the braid component 300 can have a leading loop (e.g., leading loop 402 shown in FIGS. 4A-E) where the leading loop facilitates guide tracking during deployment by interacting with guide. The leading loop can incorporate a thicker wire to aid visibility and increase strength during deployment. The thicker wire can be radiopaque and visible when imaged.

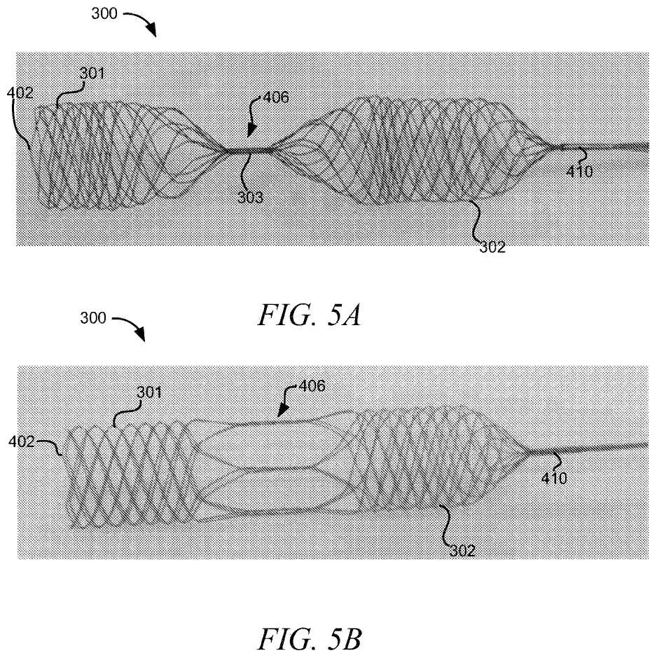

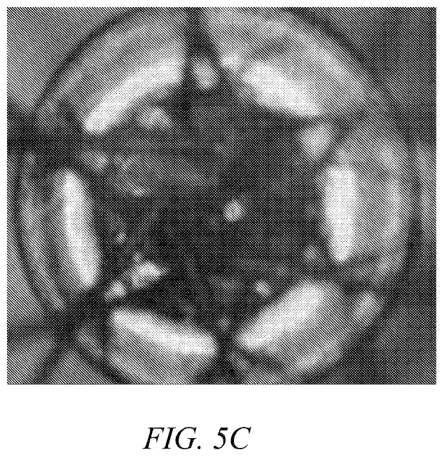

The tube, such as marker 303, can be a radiopaque marker made of platinum and iridium (Pt/Ir), gold, cobalt chrome (CoCr), stainless steel (SS), Nitinol (NiTi), any suitable radiopaque material, or any combination thereof in various possible embodiments. The tube has a length 310 and a diameter 313 where a portion of the braided wire is compacted and twisted inside the marker 303. In one embodiment, the marker 303 can be crimped onto the braided wire and positioned in a location similar to the distal neck 140 and/or nosecone of the balloon-like implant 200 where the braid waist generally lines up or is in a similar position to the balloon-like implant's 200 nosecone at implantation. The tube can include a valve mating feature where the balloon-like implant 200 has a valve that pinches or otherwise contacts the tube's valve mating feature thereby securing the balloon-like implant and tube in a known position. The valve mating feature works by lining up the waist markers with the marker in the balloon-like implant's nosecone. Once lined up, the valve closes on the braid structure, and this friction grip, along with the frontstop and backstop (discussed below with respect to FIGS. 5A-B) created by the braid slopes to the waist, can keep the braid component fixed at a known location. In this regard, the braid component's 300 proximal support portion 302 can be deployed inside the balloon-like implant 200 without shifting the vessel anchoring portion 301 of the braid component 300 where the distal anchoring portion 301 is outside of the balloon-like implant 200. The tube can constrain the braid component's 300 ability to relax, i.e., collapsing longitudally, due to the constrained sloping ends. This feature can lead to an increase in the radial force of the braid component 300 when fully deployed. It should be noted that the tube can be a small, dense braided section (e.g., braided tube 406 shown in FIGS. 4A and 4E) instead of a separate tube, such as marker 303. In this regard, the dense braid can prevent distortion, in the event the crimped marker tube offsets wires within the braid, which can cause tracking issues and unintended coiling on delivery.

The braid component 300 can be a wire braid component, which can incorporate drawn filled tubes (DFT) with a platinum, tantalum, gold, silver, or palladium core, or can be made of any other suitable material, such as a solid metal (e.g., nitinol) or flexible polymer (e.g., Polyglycolic Acid (PGA)), other radiopaque materials, or combinations thereof in various possible embodiments. The braid component's wires, or portions thereof, can be radiopaque and visible (e.g., when seen on an x-ray or similar imaging method) enabling a view of the compaction of the stent and its position during and/or after deployment. According to various embodiments, the braid component 300 is constructed from twelve or more wires. In one aspect, two to six of the wires can include a radiopaque core.

According to various embodiments, the braid component 300 can include variable pitch regions, such as pitch regions 308 and 309, where the pattern of the braid's pitch (i.e., the tightness of the wires with respect to themselves) varies. The pattern of the variable pitch regions can vary from a higher pitch to lower pitch, lower pitch to higher pitch, the same pitch, alternating pitch, can be a zebra or zig-zag pattern pitch, any suitable pitch, or combinations thereof in various possible embodiments. Tight pitch regions can have higher radial force but can have poor expansion. Open pitch regions can have better expansion but can have poor radial force. Combinations or variations of these types of regions can lead to better braid expansion, opening up the high radial force areas without coiling. Furthermore, the pitch can impact the overall softness of the braid. In this regard, varying the pitch of the braid component can create stiff and soft sections that can aid in compacting the braid component and can help the proximal portion 302 to open/deploy within the balloon-like implant 200 rather than allowing the proximal portion 302 to coil on itself during deployment.

Shown in FIGS. 4A-E, examples of the varied pitch braid component 300 can include a distal anchor portion 301, proximal support portion 302, braid waist 406 and/or marker 303, leading loop 402, compressed proximal pusher portion 404, varying pitch regions, such as pitch region 408, and varying diameters, such as diameters 414 and 416. It should be noted that any portion of the braid component 300 can include varied diameters, varied pitch wires and/or twisted wires, such as pitch region 408 shown in FIG. 4C. Twisting the wires can create stiffer sections in the braid component 300 which can assist balloon-like implant filling and/or braid compaction. Braid component 300 can include varying outer diameters, such as diameters 414 and 416, in the varied pitch regions, such as pitch regions 308, 309, and 408 shown in FIGS. 3 and 4A-E. The variable outer diameter can allow for an accordion effect, where braid rings overlap each other, creating compaction zones thereby increasing the radial force of the fully deployed braid component 300. The braid component 300 can include a flared distal tip, such as flared tip 412 shown in FIG. 4E. In some embodiments, flared distal dip 412 can also be a leading loop for interacting with the guidewire to position the braid component 300 in a vessel. The flared distal tip 412 can offset catheter deformation in the tip during tracking with the guidewire, such as guidewire 40, allowing luminal contact of the distal loops, such as leading loop 402. In this regard, migration is aided as distal loops anchor into vessel tissue resisting forward forces, such as from increasing fluid pressure of the vessel blocked by the balloon-like implant 200.

In an embodiment shown in FIG. 4E, the braid component 300 can include a compressed proximal pusher portion 404 that aids in deployment of the braid component 300 by interacting with a delivery catheter or wire to push or deliver the braid component 300 into position. A crimp, such as crimp 410, can be placed at the proximal end of the braid component 300 that holds all the braid wires together. The crimp creates a closed space, squishing all the wires together thereby securing them. In some embodiments the crimp 410 can be a Pt/Ir tube which adds radiopacity. In some embodiments crimp 410 can be stainless steel or nitinol. In some embodiments, crimp 410 is the proximal pusher portion 404. The crimp 410 can constrain the braid component's 300 ability to relax, i.e., collapsing longitudally, due to the constrained sloping ends. This feature can lead to an increase in the radial force of the braid component 300 when fully deployed.

The tube and waist features are important for at least 3 reasons: 1) The tube/waist creates a balloon-like implant aligning shape to help with deployment of the braid component distal anchor portion and proximal balloon-like implant support portion. This also helps to ensure that a defined amount of braid remains inside and outside of the balloon-like implant. A known amount of the distal anchor portion is in the biological space, and a known amount of proximal support portion is supporting the balloon-like implant. This can help to mitigate the risk of too much braid in biological space and not enough supporting the balloon-like implant inside. 2) An occlusive waist, in conjunction with a valve in the balloon-like implant's nosecone, reduces flow of fluid through the balloon-like implant and is limited, as shown in FIG. 5C, to create an effectively immediate occlusion at implantation. 3) The tube/waist constrained sloped sections inhibit the braid's ability to relax, thereby creating high radial force zones (such as a front stop and back stop) and also can increase radial force distal and proximal to the waist.

The front stop and back stop can resist lateral movement allowing for the braid to be positioned such that a known amount of braid material is in the biological space and in the balloon-like implant. The amount of braid mass in the biological space can affect migration (i.e., too little equates to not enough luminal contact which in-turn equates to not enough friction points or endothelizable anchor locations for the structure to resist blood flow forces), and the amount in the balloon-like implant can affect its ability to occlude (i.e., less braid in the balloon-like implant equates to less radial support and more potential for recanalization channels to form as the balloon-like implant luminal contact is potentially not maximized).

In an embodiment shown in FIG. 5A, marker 303 is crimped over braided waist 406 separating the distal anchor portion 301 from the proximal support portion 302 of the braid component 300. In FIG. 5A, the braid wires in waist 406 are shown in a final configuration and are encapsulated or otherwise encompassed by marker 303. The braid wires are first twisted before marker 303 is crimped, or otherwise attached, onto the wires. Shown in FIG. 5B are the twisted braid wires of waist 406 in an intermediate configuration without having been covered by marker 303. In this example, the waist 406 separates the distal anchor portion 301 from the proximal support portion 302 of the braid component 300, and the twists in the waist wires 406 are clearly shown. Wire twists can prevent braid wire offsets for tube attachment. Without the twists, tube attachment may not be possible or can be hindered. It should be noted that the twists can consists of two or more wires, can further consists of sets off twisted wires, or any combination thereof in various possible embodiments. In one embodiment, the waist comprises a 0.3 mm high density braid, shown in FIG. 5C. In this regard, a valve can seal around the high-density braid, and the friction of the valve on the wires can fix the waist to the balloon-like implant's nosecone, allowing for filling of the braid in the balloon-like implant.

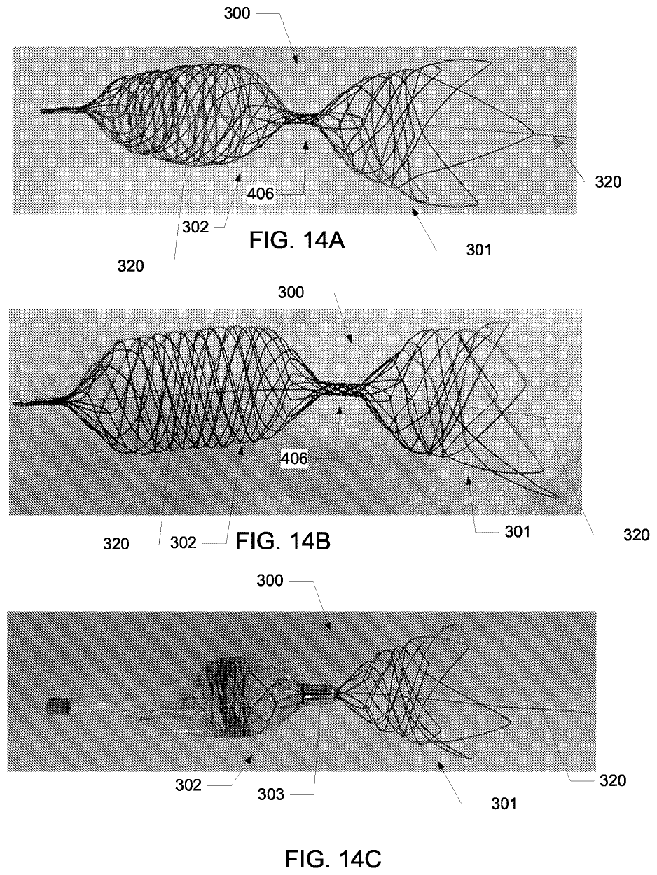

Another embodiment of an expandable body 300 is shown in FIGS. 14A-C. This embodiment includes one or more structures found in the embodiments shown in FIGS. 3-5C. For example, the expandable body includes a distal anchoring portion 301 and a proximal support portion 302, and in some embodiments a marker 303. As shown, in at least one embodiment, the expandable body 300 also includes a core wire 320 disposed along a central longitudinal axis of the expandable body. According to various aspects, the core wire 320 serves as visual deployment aid for successful deployment. The core wire 320 can be platinum or any other biocompatible and radiopaque material, including combinations thereof.

According to some embodiments, the core wire 320 does not interact with the expandable body 300 during tracking or deployment; while in other embodiments, the core wire 320 may aid in deployment of the expandable body. The core wire 320 may be cut to length based in the length of the collapsed braid or preferably the length of the balloon, as supported by the.

Shown in FIG. 6 is an example of a dual delivery system 600 with the balloon-like implant 200 shown before being deployed in its final position and inflated form. In an embodiment, the second catheter 174 is shown passing through lumens of the first catheter 173, proximal neck 130, balloon-like implant 200, and distal neck 140. It should be noted that distal neck 140 can be a distal nosecone or include a distal nosecone. In this embodiment, the proximal portion 604 of guidewire 40 passes through the lumen of a side catheter then into and through an opening, such as opening 608, which brings guidewire 40 outside of the side catheter 602 via an opening, such as opening 608, in the distal portion of the side catheter 602. In some embodiments, the opening 608 is skived at an angle ranging from 30 to 70 degrees. The outside portion 606 of guidewire 40 runs generally parallel to the first catheter 173, balloon-like implant 200, and second catheter 174. The distal end of guidewire 40 can pass through and interact with leading loop 402 of braid component 300 thereby guiding braid component 300 and/or the balloon-like implant to their desired positions. In this regard, the dual delivery system 600 can allow for both the balloon-like implant 200 and braid component 300 to be guided and delivered to their respective locations at the same time. Once the balloon-like implant 200 and braid component 300 are guided to their desired positions, the guidewire 40 can be retracted and deployment of the balloon-like implant 200 and braid component 300 can proceed. In one embodiment, the deployment of the balloon-like implant 200 and braid component 300 can proceed without retracting the guidewire 40 from the side catheter 602. It should be noted that guidewire 40 can be a microcatheter, tube, wire, or other guide capable of guiding the balloon-like implant and/or the expandable device, such as braid component 300, over the guide path.

The walls 30 of the detachable balloon-like implants can comprise one or more layers. The thickness of the walls 30 can range between 5-400 microns or between 0.0002-0.016 in.

Some detachable balloon-like implant 10 sizes and shapes are better suited for the treatment of some conditions while others are better suited for the treatment of other conditions. For example, a rounded or spherical detachable balloon-like implant 10 can be better suited for treating saccular aneurysms and LAAs. In contrast, a cylindrical detachable balloon-like implant can be better suited for treating arteries 317, as shown in FIG. 12A; veins 318, as shown in FIG. 12B, paravalvular leaks and biological conduits. For other clinical applications, detachable balloon-like implants can be configured to assume an expanded shape comprising two or more lobes, excluding proximal and distal necks 130 & 140 and neck assemblies, if any.

In some embodiments, all or a portion of a detachable balloon-like implant 10 of a detachable balloon catheter 1 is non-compliant. In some embodiments, all or a portion of a detachable balloon-like implant 10 of a detachable balloon catheter 1 is semi-compliant. In some embodiments, all or a portion of a detachable balloon-like implant 10 of a detachable balloon catheter 1 is compliant. In some embodiments, all or a portion of a detachable balloon-like implant 10 of a detachable balloon catheter 1 grows <2%, <4%, <6%, <8%, <10%, or >10% when inflated to a pressure in a range of 1-20 atmospheres.

In some embodiments, the detachable balloon-like implant 10 comprises an opening in the proximal region 110 to enable fluid to pass from the catheter or catheter assembly 5 into the detachable balloon-like implant 10. In some embodiments, the proximal opening in the detachable balloon-like implant 10 further comprises a proximal neck 130 that extends away from the detachable balloon-like implant 10 or extends into the central void 115 of the detachable balloon-like implant 10, as shown in FIGS. 1A-B.

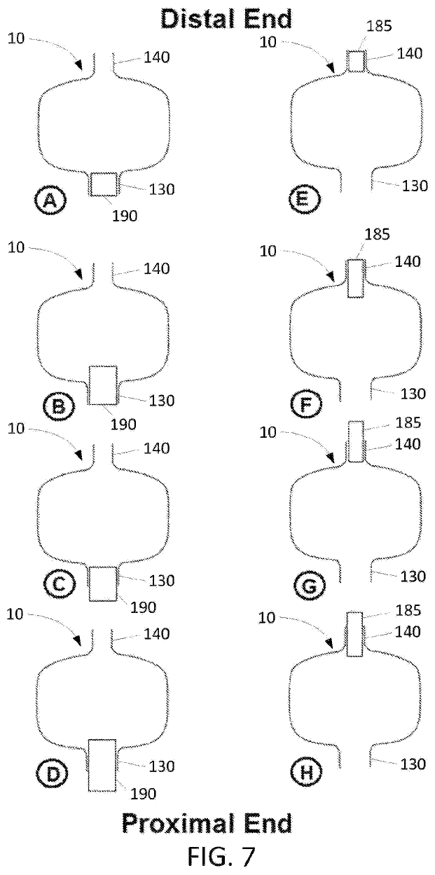

As shown in FIGS. 7A-D, one or more ring structures, tubular structures, telescoping structures, catheter segments, or telescoping catheter segments can be joined to the proximal neck 130 of the detachable balloon-like implant 10. Such a structure is called a “proximal telescope” 190 and, along with the proximal neck 130, forms a proximal neck assembly.

Various configurations of proximal telescopes 190 can be employed to achieve various embodiments of a proximal neck assembly. The proximal telescope 190 can be longer than, shorter than, or the same length as the proximal neck 130 of the detachable balloon-like implant 10. The proximal telescope 190 can project distal to, proximal to, both distal and proximal to, or neither distal nor proximal to, the proximal neck 130. The outer surface of the proximal telescope 190 can be joined to the inner surface of the proximal neck 130. The inner surface of the proximal telescope 190 can be joined to the outer surface of the proximal neck 130. The proximal telescope 190 can be joined to the proximal neck 130 with an adhesive or glue. The proximal telescope 190 can be rigid, can comprise a metal, or can comprise a radiopaque metal that is visible during fluoroscopy. A metal proximal telescope 190 can comprise platinum, iridium, gold, silver, stainless steel, nitinol, titanium, or alloys or combinations thereof. The proximal telescope 190 can be flexible or can comprise a polymer. A polymer proximal telescope 190 can comprise Pebax, nylon, polyimide, PTFE, or combinations thereof. A polymer proximal telescope 190 can comprise a polymer or polymers with a Shore durometer hardness of 20-80 D. The inner layer of the wall of a polymer proximal telescope 190 can comprise a lubricious polymer including, but not limited to, PTFE, polyimide, a composite, or a mixture of polyimide and PTFE. The wall of a polymer proximal telescope 190 can comprise a middle layer located between an outer layer and an inner layer, such middle layer comprising metal wire, including metal wire comprising nitinol or stainless steel, and including metal wire configured in a spiral, coil, braid, woven, or straight pattern. The proximal telescope 190 can comprise a lubricious coating on its inner surface, outer surface, or both inner and outer surfaces. The proximal telescope 190 can comprise a hydrophilic coating.

The proximal telescope 190 can comprise a marker band that is conspicuous during fluoroscopy. The marker band can comprise platinum, iridium, gold, silver, or alloys or combinations thereof. A marker band can be joined to the distal, proximal, or both the proximal and distal ends of the proximal telescope 190.

Various dimensions of proximal telescopes 190 can be specified to achieve various embodiments of a proximal neck assembly. The internal or luminal diameter of the proximal telescope 190 can be 0.024-0.108 inch. The external or overall diameter of the proximal telescope 190 can be 0.026-0.110 inch. The proximal telescope 190 can have a length of 0.3-30 mm either prior to or after separation of the detachable balloon-like implant 10 from the first catheter 173, as measured parallel to the first axis 706. The internal diameter of the proximal neck 130 or proximal neck assembly of the detachable balloon-like implant 10 can be 0.026-0.110 inch, as measured parallel to the second axis 708. The external diameter of the proximal neck 130 or proximal neck assembly of the balloon-like implant can be 0.032-0.130 inch, as measured parallel to the second axis 708.

The proximal neck assembly of detachable balloon-like implants 10 can further comprise a proximal nosecone to reduce the risk of injury to the wall of a saccular aneurysm, artery 317, vein 318, LAA, other blood-containing structure, biological conduit, or biological space when advancing or retracting the detachable balloon catheter 1. Although not illustrated, a proximal nosecone can be structurally and functionally similar to the distal nosecone. The proximal nosecone can have a tapered proximal end, a tapered distal end, or tapered proximal and distal ends. In some embodiments, the proximal nosecone comprises one piece, while in other embodiments, the proximal nosecone comprises two or more pieces that are bonded together, including bonded together with a glue or adhesive. In some embodiments, the proximal nosecone comprises one or more polymers, including polyether ether ketone (PEEK), polycarbonate, nylon, polyimide, Pebax, PTFE, silicone, polyurethane, co-polyester polymer, thermoplastic rubber, silicone-polycarbonate copolymer, polyethylene ethyl-vinyl-acetate (PEVA) co-polymer, a biocompatible elastomer, biocompatible resilient material, or a biocompatible adhesive. In some embodiments, the length of the proximal nosecone is 1-10 mm. In some embodiments, the proximal nosecone has an outer diameter of 0.054-0.18 inch. In some embodiments, the proximal nosecone is bonded to a proximal neck 130. In some embodiments, a proximal nosecone is bonded to a portion of a proximal neck assembly, including a proximal telescope 190. In some embodiments, a proximal nosecone is bonded to both a proximal neck 130 and a portion of a proximal neck assembly. In some embodiments, a portion of the inner surface of a proximal nosecone is bonded to a portion of the outer surface of a proximal balloon-like implant neck 130 or a portion of a proximal neck assembly. In some embodiments, at least a portion of the proximal neck 130 comprises a layer of radiopaque metal that is visible under fluoroscopy.

In some embodiments, the detachable balloon-like implant comprises an opening in the distal region 120 to enable a portion of a catheter or catheter assembly 5 to pass into and through the central void 115 of the detachable balloon-like implant 10 and optionally extend distal to the detachable balloon-like implant 10, thereby enabling a guidewire 40 and or second catheter 174 to pass completely through the detachable balloon-like implant 10. In some embodiments, the distal opening in detachable balloon-like implant 10 further comprises a distal neck 140 that extends away from the detachable balloon-like implant 10 or extends into the central void 115 of the detachable balloon-like implant 10, as shown in FIGS. 1A-B.

As shown in FIGS. 7E-H, one or more ring structures, tubular structures, telescoping structures, catheter segments, or telescoping catheter segments can be joined to the distal neck 140 of the detachable balloon-like implant 10. Such a structure is called a “distal telescope” 185 and, along with the distal neck 140, forms a distal neck assembly.

Various configurations of distal telescopes 185 can be employed to achieve various embodiments of a distal neck assembly. The distal telescope 185 can be longer than, shorter than, or the same length as the distal neck 140 of the detachable balloon-like implant 10. The distal telescope 185 can project proximal to, distal to, both distal and proximal to, or neither distal nor proximal to, the distal neck 140. The outer surface of the distal telescope 185 can be joined to the inner surface of the distal neck 140. The inner surface of the distal telescope 185 can be joined to the outer surface of the distal neck 140. The distal telescope 185 can be joined to the distal neck 140 with an adhesive or glue. The distal telescope 185 can be rigid, can comprise a metal, or can comprise a radiopaque metal that is visible during fluoroscopy. A metal distal telescope 185 can comprise platinum, iridium, gold, silver, stainless steel, nitinol, titanium, or alloys or combinations thereof. The distal telescope 185 can be flexible or can comprise a polymer. A polymer distal telescope 185 can comprise Pebax, nylon, polyimide, PTFE, or combinations thereof. A polymer distal telescope 185 can comprise a polymer or polymers with a Shore durometer hardness of 20-80 D. The inner layer of the wall of a polymer distal telescope 185 can comprise a lubricious polymer including, but not limited to, PTFE, polyimide, a composite, or a mixture of polyimide and PTFE. The wall of a polymer distal telescope 185 can comprise a middle layer located between an outer layer and an inner layer, such middle layer comprising metal wire, including metal wire comprising nitinol or stainless steel, and including metal wire configured in a spiral, coil, braid, woven, or straight pattern. The distal telescope 185 can comprise a lubricious coating on its inner surface, outer surface, or both inner and outer surfaces. The distal telescope 185 can comprise a hydrophilic coating.

The distal telescope 185 can comprise a marker band that is conspicuous during fluoroscopy. The marker band can comprise platinum, iridium, gold, silver, or alloys or combinations thereof. A marker band can be joined to the distal, proximal, or both the proximal and distal ends of the distal telescope 185.

Various dimensions of distal telescopes 185 can be specified to achieve various embodiments of a distal neck assembly. The internal or luminal diameter of the distal telescope 185 can be 0.024-0.108 inch. The external or overall diameter of the distal telescope 185 can be 0.026-0.110 inch. The distal telescope 185 can have a length of 0.3-30 mm either prior to or after separation of the detachable balloon-like implant 10 from the first catheter 173, as measured parallel to the first axis 706. The internal diameter of the distal neck 140 or distal neck assembly can be 0.024-0.110 inch, as measured parallel to the second axis 708. The external diameter of the distal neck 140 or distal neck assembly can be 0.026-0.110 inch, as measured parallel to the second axis 708.

In some embodiments of a detachable balloon catheter 1, the outer diameter of the distal telescope 185 is greater than the internal diameter of proximal telescope 190, such that the proximal portion of the distal telescope 185 cannot enter the first lumen 162. In some embodiments of a detachable balloon catheter 1, the outer diameter of the distal telescope 185 is greater than the internal diameter of the proximal neck 130, such that the proximal portion of the distal telescope 185 cannot enter the first lumen 162. In some embodiments of a detachable balloon catheter 1, the outer diameter of the distal telescope 185 is greater than the internal diameter of the first catheter 173, such that the proximal portion of the distal telescope 185 cannot enter the first lumen 162.

The distal neck assembly of detachable balloon-like implants 10 can further comprise a distal nosecone to reduce the risk of injury to the wall of a saccular aneurysm, artery 317, vein 318, LAA, other blood-containing structure, biological conduit, or biological space when advancing or retracting the detachable balloon catheter 1. The distal nosecone can have a tapered proximal end, a tapered distal end, or tapered proximal and distal ends. In some embodiments, the distal nosecone comprises one piece, while in other embodiments, the distal nosecone comprises two or more pieces that are bonded together including bonded together with a glue or adhesive. In some embodiments, the distal nosecone comprises one or more polymers, including polyether ether ketone (PEEK), polycarbonate, nylon, polyimide, Pebax, PTFE, silicone, polyurethane, co-polyester polymer, thermoplastic rubber, silicone-polycarbonate copolymer, polyethylene ethyl-vinyl-acetate (PEVA) co-polymer, a biocompatible elastomer, biocompatible resilient material, or a biocompatible adhesive. In some embodiments, the length of the distal nosecone is 1-10 mm. In some embodiments, the distal nosecone has an outer diameter of 0.058-0.18 inch. In some embodiments, the distal nosecone is bonded to a distal neck 140. In some embodiments, a distal nosecone is bonded to a portion of a distal neck assembly, including a distal telescope 185. In some embodiments, a distal nosecone 191 is bonded to both a distal neck 140 and a portion of a distal neck assembly. In some embodiments, a portion of the inner surface of a distal nosecone is bonded to a portion of the outer surface of a distal balloon-like implant neck 140 or a portion of a distal neck assembly. In some embodiments, at least a portion of the distal neck 140 comprises a layer of radiopaque metal that is visible under fluoroscopy.

Detachable balloon-like implants can include rigid materials, semi-rigid materials, polymer materials, metal or metalized materials, or combinations thereof in various possible embodiments. In some embodiments, detachable balloon-like implants 10 can be polymer balloon-like implants 12, wherein they comprise a continuous layer of polymer, excluding any proximal and distal openings in the detachable balloon-like implant 10. The continuous polymer layer 99 of detachable polymer balloon-like implants 12 can comprise a non-compliant material, such as but not limited to nylon or Pebax. Preferably, the non-compliant material is tear-resistant and can be manufactured into thin-walled structures. The thickness of the polymer layer of a detachable polymer balloon-like implant 12 can range between 5-300 microns or between 0.0002-0.012 inch. Detachable polymer balloon-like implants 12 can comprise additional layers of non-metallic coatings or polymers, which can be continuous or discontinuous, and which can be internal to the continuous polymer layer or external to the continuous polymer layer.

The additional layers of non-metallic coatings or polymers can comprise polyurethane, silicone, or poly(p-xylylene) (Parylene). The additional layers of non-metallic coatings or polymers of detachable polymer balloon-like implants 12 can have a thickness of 0.1 100 microns. The overall thickness of the wall 30 of detachable polymer balloon-like implants 12 can range between 5-300 microns, or between 0.0002-0.012 inch.

Detachable polymer balloon-like implants 12 may not possess sufficient strength to maintain an expanded or partially expanded configuration in vivo after separation from a catheter or catheter assembly 5. Detachable polymer balloon-like implants 12 may not possess sufficient strength to maintain an expanded or partially expanded configuration in vivo after separation from a catheter or catheter assembly 5 when no solid or semi-solid material, not derived from the patient, is present in the central void 115 of the expanded detachable polymer balloon-like implant after separation from the first and second catheters 173 & 174. Detachable polymer balloon-like implants 12 may not possess sufficient strength to maintain an expanded or partially expanded configuration in vivo after separation from a catheter or catheter assembly 5 when the detachable polymer balloon-like implant 12 is implanted in an unsealed configuration. Detachable polymer balloon-like implants 12 may not possess sufficient strength to maintain an expanded or partially expanded configuration in vivo after separation from a catheter or catheter assembly 5 when the pressure in the central void 115 or interior volume of the expanded detachable polymer balloon-like implant 12 is not greater than a pressure outside the expanded detachable polymer balloon-like implant 12. By using molds and balloon blowing techniques, detachable polymer balloon-like implants 12 that closely match the size and shape of various vascular structures, biological conduits, or biological spaces can be made, including but not limited to saccular aneurysms, segments of arteries 317, segments of veins 318, LAAs, paravalvular leak paths, segments of biological conduits, or particular biological spaces.

In some embodiments, all or a portion of a detachable polymer balloon-like implant 12 of a detachable balloon catheter 1 is non-compliant. In some embodiments, all or a portion of a detachable polymer balloon-like implant 12 of a detachable balloon catheter 1 is semi-compliant. In some embodiments, all or a portion of a detachable polymer balloon-like implant 12 of a detachable balloon catheter 1 is compliant. In some embodiments, all or a portion of a detachable polymer balloon-like implant 12 of a detachable balloon catheter 1 grows <2%, <4%, <6%, <8%, <10%, or >10% when inflated to a pressure in a range from 1 to 20 atmospheres.

In some embodiments, the external surface of a polymer detachable balloon-like implant 12 of a detachable balloon catheter 1 comprises surface structures.

In some embodiments, the external surface of a polymer detachable balloon-like implant 12 of a detachable balloon catheter 1 comprises surface structures having a height of 0.01-1 microns. In some embodiments, the external surface of the proximal region 110 of a polymer detachable balloon-like implant 12 of a detachable balloon catheter 1 comprises surface structures or surface structures having a height of 0.01-1 microns, while the intermediate region 100 and distal region 120 are smooth or smoother than the proximal region 110. In some embodiments, the external surface of the proximal region 110 of a polymer detachable balloon-like implant 12 of a detachable balloon catheter 1 configured for implantation into a saccular aneurysm or LAA comprises surface structures or surface structures having a height of 0.01-1 microns, while the intermediate region 100 and distal region 120 are smooth or smoother than the proximal region 110.

Detachable balloon-like implants 10 can further comprise an expandable metal retention structure 731 to reduce the risk of migration after placement in the lumen of a segment of a vein 318, a LAA, or other blood-containing structure, biological conduit or biological space, as shown in FIG. 12B. Such a feature could provide an additional factor of safety when filling or occluding a vein segment 318, for example, because the lumen diameter of veins generally increases in the direction of flow. Therefore, device migration in veins 318 is not self-limiting and the device can reach the right atrium, right ventricle, or a branch of a pulmonary artery branch, potentially leading to the symptoms of a pulmonary embolism. In contrast, the lumen diameter of arteries 317 generally decreases in the direction of flow. Therefore, device migration in arteries 317 is self-limiting and the use of an expandable metal retention structure 731 can be less critical when filling or occluding an artery segment 317 with a detachable balloon-like implant 10, as shown in FIG. 12A.

After expansion, the diameter of a portion of the metal retention structure 731 is equal to or greater than the diameter of the expanded detachable balloon-like implant 10. As shown in FIG. 12B, a portion of the expandable metal retention structure 731 is configured to make contact with the wall of an artery 317, vein 318, LAA, aneurysm, biological conduit, or other blood containing space or biological space. The expandable metal retention structure 731 comprises a plurality of elongated ribs or elongated arms 730. The expandable metal retention structure 731 can comprise a plurality of elongated ribs extending from both a proximal retention ring and a distal retention ring 9606, as shown in FIGS. 12B and 12F.

In some embodiments, the expandable metal retention structure 731 is self-expanding. The retention structure 731 can comprise nitinol, cobalt chrome, or stainless steel. The external diameter of the ring structures when expanded, is in a range from 3-45 mm, and the diameter of the detachable balloon-like implant 10, when expanded, is 3-40 mm.

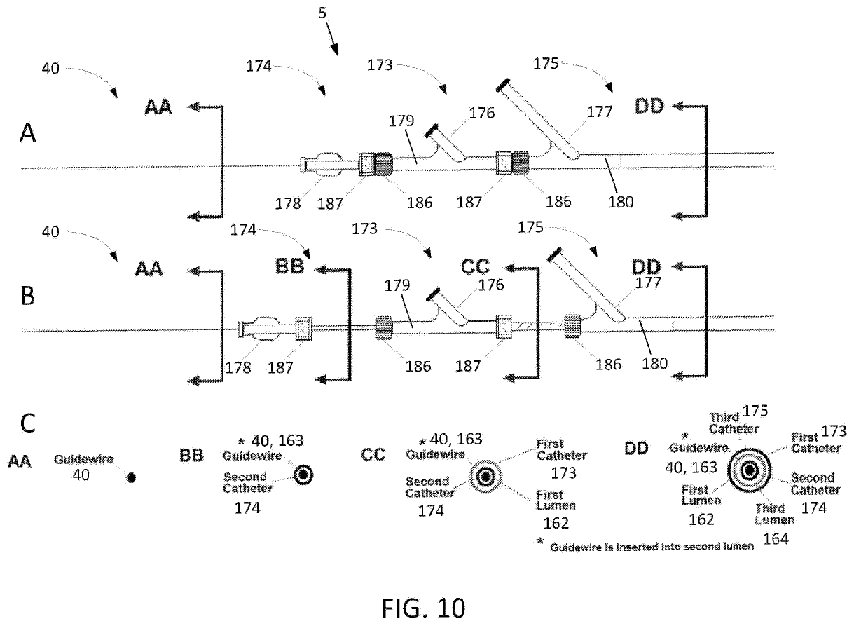

Detachable balloon catheters 1 comprising an expandable metal retention structure 731 can further comprise an outer catheter (also called a “third catheter”) 175, as shown in FIGS. 10A-C, wherein a distal portion of the third catheter 175 passes over at least a portion of the expandable retention structure 731 and retains the expandable retention structure 731 in a constrained, compressed, or collapsed configuration.

Shown in FIGS. 12C-D and in some embodiments, the retention structure 731 can be a braid component 300 or portion thereof, such as anchor portion 301. When deployed, the anchor portion 301 can self-expand to contact at least a portion of the biological space where wires of the braid can hold the balloon-like implant 10 in a position so as not to move when pressures, such as pressure from blood flow, press on the balloon-like implant 10. In one embodiment, the anchor portion 301 interacts with a guidewire 40 or microcatheter to expand and contact a surface of the biological space to anchor the balloon-like implant 10 in position. The interaction with guidewire 40 can be facilitated by a leading loop 402 of the anchor portion 301 where the guidewire 40 guides the anchor portion 301 into a desired location. In some embodiments, the braid component 300 is separate from the balloon-like implant 10 but can interact with balloon-like implant 10 where a portion of the braid component 300, such as a proximal support portion 302, is deployed within the balloon-like implant 10 to support and/or anchor the balloon-like implant 10. After the distal anchor portion 301 of the braid component 300 is deployed, the proximal support portion 302 can be deployed or otherwise positioned inside balloon-like implant 10. In this regard, the support portion 302 is expanded to contact the inner wall 30 of balloon-like implant 10 to support and/or position the balloon-like implant thereby aiding the balloon-like implant to remain in a desired position. In some embodiments, the proximal support portion 302 of braid component 300 can be self-expanding where the wires are formed in a conical, twisted, or conical form that naturally spring into a final, fully expanded shape. In some embodiments, the support portion 302 can require interaction with a guidewire, microcatheter, or catheter to achieve its final, fully expanded shape and may not be self-expanding. Regions, such as pitch region 408, of the support portion 302 can vary in pitch and/or diameter thereby creating stiffer sections that inhibit the support portion 302 from collapsing or expanding on itself.

In some embodiments, the external surface of the detachable balloon-like implant 10 comprises surface structures. In certain instances, these surface structures increase surface roughness, increase frictional forces between the external surface of a detached balloon-like implant and the internal surface of a saccular aneurysm, artery 317, vein 318, LAA, paravalvular leak path, other blood-containing structure, or biological conduit or space, thereby reducing the risk of movement or migration of the detachable balloon-like implant 10 following its deployment. In some embodiments, the surface structures have a height of 0.01-1 micron. In some embodiments, the exterior surface of the detachable balloon-like implant 10 comprises a rounded, pebbled, or granular structure.

In some embodiments, the external surface of the detachable balloon-like implant 10 comprises a lubricous coating. In certain instances, this lubricous or hydrophilic coating reduces the frictional forces between the external surface of a detachable balloon-like implant 10 and the internal surface of a saccular aneurysm, artery 317, vein 318, LAA, paravalvular leak path, other blood-containing structure, or biological conduit or space, thereby reducing the risk of tissue injury during placement and expansion of a detachable balloon-like implant 10. In some embodiments, the lubricous or hydrophilic coating is a hydrophilic coating, a Serene™ coating that is durable.

### Catheter Assemblies

The present disclosure relates to embodiments of a first medical device 1 comprising a detachable balloon-like implant 10 and a catheter or catheter assembly 5; as shown in in FIGS. 8A-D, 9A-C, and 10A-C; wherein the detachable balloon-like implant 10 is configured for expansion with fluid and detachment from the catheter or catheter assembly 5 in vivo.

The first catheter 173 of the detachable balloon catheter 1 has a proximal end, a lumen configured to accept a second catheter 174, and a distal end that is joined or operably coupled to the proximal region 110 of the detachable balloon-like implant 10, as shown in in FIG. 8C, and 9A-C. In various embodiments, the first catheter 173 is joined to a portion of the proximal neck 130 of a detachable balloon-like implant 10, joined to a portion of the proximal neck assembly of the detachable balloon-like implant 10, or joined to a tubular segment that is interposed between the first catheter 173 and the proximal neck 130 or proximal neck assembly of the detachable balloon-like implant 10.

In some embodiments, an outer layer of the wall of the first catheter 173 comprises a polymer, or comprises Pebax, nylon, polyimide, and PTFE. In some embodiments, an inner layer of the wall of the first catheter 173 comprises a lubricious polymer or comprises PTFE, polyimide, a composite, or mixture of polyimide and PTFE. In some embodiments, the first catheter 173 includes a middle layer comprising metal, wherein the middle layer is located between an outer layer and an inner layer. The metal of the middle layer of the first catheter 173 can be configured as wire, including metal or wire configured in a spiral, coil, braid, woven, or straight pattern, or combinations thereof. In some embodiments, the metal or metal wire comprises nitinol or stainless steel. In some embodiments, the wire is round and has a diameter of 0.0005-0.0030 inch. In some embodiments, the wire is configured in a coil with a pitch of 0.0010-0.0060 inch. In some embodiments, the wire is flat and has a thickness of 0.0005-0.0060 inch and a width of 0.001-0.030 inch. In some embodiments, wherein the wire is configured in a braid, the braid has a picks per inch of length (PPI) of 50-300, and in some embodiments, the wire is wound in a braid in an “under one, over two” pattern. In some embodiments, the proximal portion of the wire in the first catheter 173 is flat, has a thickness of 0.0005-0.0060 inch and a width of 0.001-0.030 inch, and is configured in a braid configuration with a picks per inch of length of 50-300; and wherein the wire in the distal portion of the first catheter 173 is round, has a diameter of 0.0005-0.0030 inch, and is configured in a coil pattern with a pitch of 0.0010-0.0080 inch.

In some embodiments, the first catheter 173 comprises a lubricious or hydrophilic coating, including a lubricious or hydrophilic coating that is present on the inner surface, the outer surface, or both the inner and outer surface of the first catheter 173. In some embodiments, the lubricious or hydrophilic coating is present on the distal portion of the first catheter 173, but absent from the proximal portion of the first catheter 173.

In some embodiments, the outer layer of the proximal end of the first catheter 173 comprises a material with a Shore durometer hardness of 40-90 D. In some embodiments, the outer layer of the proximal end of the first catheter 173 comprises nylon. In some embodiments, the outer layer of the distal end of the first catheter 173 comprises a material with a Shore durometer hardness of 20-60 D. In some embodiments, the outer layer of the distal end of the first catheter 173 comprises nylon, urethan, Pebax, and combinations of the same.

In some embodiments, the internal or luminal diameter of the first catheter 173 is 0.025-0.068 inch. In some embodiments, the external diameter of the first catheter 173 is 0.031-0.096 inch. In some embodiments, the length of the first catheter 173 is 45-245 cm.

In some embodiments, the wall of the first catheter 173 is continuous from the proximal end to the distal end. In one example, the outer layer of the proximal portion of the first catheter 173 comprises a nylon with a Shore durometer hardness of 40-90 D, the middle portion of the first catheter 173 comprises Pebax with a Shore durometer hardness of 20-60 D, and the distal portion of the first catheter 173 comprises nylon with a Shore durometer hardness of 40-90 D. In some embodiments, the distal portion has a hardness of 20-40 D and comprises Pebax or urethane and the middle portion has a hardness of 30-60 D. The distal portion of the first catheter 173 is joined to a portion of the proximal neck 130 or proximal neck assembly of the balloon-like implant 10 by a friction fit formed between the outer surface of the first catheter 173 and an elastomeric tubular segment joined or bonded to the proximal neck 130 or proximal neck assembly of the detachable balloon-like implant. In some embodiments, the first catheter 173 is joined by a superelastic tubular segment. In some embodiments, the distal portion of the first catheter 173 or first catheter assembly of a detachable balloon catheter 1 is coupled to the distal neck 140 or distal neck assembly of the detachable balloon-like implant and the wall of the segment of the first catheter 173 that passes through the central void 115 or interior volume of the detachable balloon-like implant 10 comprises openings for fluid to pass out of the lumen 163 of the second catheter 174. Although not illustrated, this distally joined configuration is structurally and functionally similar to the proximally joined configuration mentioned above. In some embodiments, the proximal hub of the first catheter 173 or first catheter assembly of a detachable balloon catheter 1 comprises a port for the injection of fluids (also called an “inflation port”) 176 into the first lumen (also called an “inflation lumen”) 162, as shown in FIGS. 8C and 9A-C.

In some embodiments, the second catheter 174 of the detachable balloon catheter 1 has a proximal end, a distal end that is open, and a lumen 163 configured to accept a guidewire 40 (shown in FIGS. 8B and 9A-C), one or more expandable body, other medical devices, or combinations thereof. In some embodiments, an outer layer of the wall of the second catheter 174 comprises a polymer, or comprises Pebax, nylon, polyimide, or PTFE. In some embodiments, an inner layer of the wall of the second catheter 174 comprises a lubricious polymer, or comprises PTFE, polyimide, a composite, or mixture of polyimide and PTFE. In some embodiments, the second catheter 174 includes a middle layer comprising metal, wherein the middle layer is located between an outer layer and an inner layer. In some embodiments, the middle layer comprising metal is configured as wire. In some embodiments, the middle layer comprising metal is configured in one or more of a spiral, coil, braid, woven, or straight pattern. In some embodiments, the middle layer comprises nitinol or stainless steel. In some embodiments, the wire of the middle layer is round and has a diameter of 0.0005-0.0030 inch. In some embodiments, the wire of the middle layer is configured in a coil with a pitch of 0.0010-0.0060 inch. In some embodiments, the wire of the middle layer is flat and has a thickness of 0.0005-0.0060 inch and a width of 0.001-0.030 inch. In some embodiments, the wire of the middle layer is configured in a braid with a picks per inch of length (PPI) of 50-300, or configured in a braid that is wound in an “under one, over two” pattern. In one example, the wire in the middle layer of the proximal portion of the second catheter 174 is flat, has a thickness of 0.0005-0.0060 inch and a width of 0.001-0.030 inch, and is configured in a braid configuration with a picks per inch of length of 50-300; and the wire in the middle layer of the distal portion of the second catheter 174 is round, has a diameter of 0.0005-0.003 inch, and is configured in a coil pattern with a pitch of 0.0010-0.0060 inch. In some embodiments, the second catheter 174 comprises a lubricious or hydrophilic coating, or comprises a lubricious or hydrophilic coating on the inner surface, the outer surface, or both the inner and outer surface of the second catheter 174. In some embodiments, a lubricious or hydrophilic coating is present on the distal portion of the second catheter 174, but absent from the proximal portion of the second catheter 174. In some embodiments, the proximal end of the second catheter 174 comprises a material with a Shore durometer hardness of 40-90 D. In some embodiments, the outer layer of the proximal end of the second catheter 174 comprises nylon. In some embodiments, the outer layer of the proximal end of the second catheter 174 comprises nylon with a Shore durometer hardness of 40-90 D. In some embodiments, the distal end of a second catheter 174 comprises a material with a Shore durometer hardness of 20-60 D, urethane, or Pebax with a Shore durometer hardness of 20-60 D. In some embodiments, the distal end of the second catheter 174 comprises a material with a Shore durometer hardness of 40-90 D, or nylon with a Shore durometer hardness of 40-90 D. In some embodiments, the second catheter 174 comprises at least two marker bands that are conspicuous during fluoroscopy and are configured to assist in the passage of coils, expandable bodies, or other medical devices through the lumen of the second catheter 174 and to assist the detachment of coils, expandable bodies, or other medical devices that are passed through the lumen of the second catheter 174. In some embodiments, a first marker band is 0.3-1.5 mm from the distal end of the second catheter 174 and a second marker band is 2.0-4.0 mm proximal to the distal end of the second catheter 174. In some embodiments, the second marker band is 26-32 mm from the distal end of the second catheter 174 or 28-32 mm from the distal-most marker band. In some embodiments, the internal or luminal diameter of the second catheter 174 is 0.016-0.068 inch and a corresponding external diameter can be 0.031-0.088 inch. In some embodiments, the internal or luminal diameter of the second catheter 174 is 0.012-0.048 inch. In some embodiments, the external diameter of the second catheter 174 is 0.018-0.068 inch. In some embodiments, the second catheter 174 comprises a hub and a shaft. In some embodiments, the length of the second catheter 174 is 50 -250 cm. In some embodiments, the wall of the second catheter 174 is continuous from the proximal end to the distal end. In some embodiments of a detachable balloon catheter 1, the wall of a segment of the second catheter 174 that passes through the central void 115 or interior volume of the balloon-like implant comprises openings for fluid to pass out of the lumen of the second catheter 174. In one example, the outer layer of the proximal portion of the second catheter 174 comprises a material with a Shore durometer hardness of 40-90 D and the outer layer of the distal end of the second catheter 174 comprises a material with a Shore durometer hardness of 40-90 D, and wherein the distal end of the second catheter 174 comprising a material with a Shore durometer hardness of 20-60 D is interposed with a segment of material with a Shore durometer hardness of 40-90 D. In another example, the outer layer of the proximal portion of the second catheter 174 comprises nylon with a Shore durometer hardness of 40-90 D and the outer layer of the distal end of the second catheter 174 comprises Pebax with a Shore durometer hardness of 20-60 D, and wherein the Pebax at the distal end of the second catheter 174 is interposed with a segment of nylon with a Shore durometer hardness of 40-90 D.

In some embodiments, the distal portion of a second catheter 174 of a detachable balloon catheter 1 is joined or operably coupled to a portion of the one or more elastomeric valves by a friction fit. In some embodiments, the distal portion of a second catheter 174 of a detachable balloon catheter 1 is joined or operably coupled to a portion of the one or more elastomeric valves by a friction fit, wherein the elastomeric valves are contained within a distal nosecone bonded to the distal neck 140 or the distal neck assembly of the detachable balloon-like implant. In some embodiments, the distal portion of a second catheter 174 of a detachable balloon catheter 1 is joined or operably coupled to a portion of the one or more elastomeric valves and spacers by a friction fit. In some embodiments, the distal portion of a second catheter 174 of a detachable balloon catheter 1 is joined or operably coupled to a portion of the one or more elastomeric valves or spacers by a friction fit, wherein the elastomeric valves and spacers are contained within a distal nosecone bonded to the distal neck 140 or the distal neck assembly of the detachable balloon-like implant. In some embodiments, the distal portion of a second catheter 174 of a detachable balloon catheter 1 is joined or operably coupled to a portion of the one or more elastomeric valves by a friction fit, wherein the one or more elastomeric valves and the one or more spacers overlap the segment of material in the second catheter 174 with a Shore durometer hardness of 40-90 D that is interposed into the material in the second catheter 174 with a Shore durometer hardness of 20-60 D. In some embodiments, the distal portion of the second catheter 174 of the detachable balloon catheter 1 is angled. In some embodiments, the angle between the distal portion of the second catheter 174 and the proximal portion of the second catheter 174 of the detachable balloon catheter 1 is 1-70 degrees. In some embodiments, the proximal hub of the second catheter 174 of a detachable balloon catheter 1 is configured for the injection of fluids into the second lumen.

In some embodiments, the color of the external surface of a first 173, second 174, or third 175 catheter is configured to help physicians use the device safely. In some embodiments, the color of the most proximal portion of the second catheter 174 could be of one color (the first color), while the more distal portion of the second catheter 174 could be another color (the second color). The length of the most proximal portion would be chosen to correspond to the length from the distal end of the second catheter 174 (as packaged) to the distal end of the male tubular structure 510 or from the distal end of the second catheter 174 (as packaged) to a location 1-10 mm distal to the distal end of the male tubular structure 510, such that while retracting the second catheter 174, if the physician sees the second color then stops retracting to avoid inadvertent detachment of the balloon-like implant 510. In some embodiments, a flexible elongated structure could be joined to the hub 179 of the first catheter 173 and the hub 178 of the second catheter 174, such that the extended length of the flexible elongated structure could correspond to the length from the distal end of the second catheter 174 (as packaged) to the distal end of the male tubular structure, or from the distal end of the second catheter 174 (as packaged) to a location 1-10 mm distal to the distal end of the male tubular structure 510, such that while retracting the second catheter 174, the flexible elongated structure prevents the physician from retracting the second catheter 174 too far, risking inadvertent detachment of the balloon-like implant 510. To proceed with detachment, the physician could first remove, disconnect, or cut the flexible elongated structure and then retract the second catheter 174 further.

The third catheter 175 of the detachable balloon catheter 1 has a proximal end, a distal end that is open, and a lumen 164 configured to accept a first catheter 173. The inner surface of the third catheter 175 and the outer surface of the first catheter 173 define a third lumen 164 to allow for passage of fluid from the proximal hub 180 of the third catheter 175 to the distal end of the third catheter 175 and into the space adjacent to the distal end of the third catheter 175. In some embodiments, the third lumen 164 is configured for the injection of fluids, including water, saline, radiographic contrast, solutions comprising therapeutic agents or drugs, and mixtures therein. In some embodiments, an outer layer of the wall of the third catheter 175 comprises a polymer, Pebax, nylon, polyimide, or PTFE. In some embodiments, an inner layer of the wall of the third catheter 175 comprises a lubricious polymer, PTFE, polyimide, or a composite, or mixture of polyimide and PTFE. In some embodiments, the third catheter 175 comprises a middle layer comprising metal, wherein the middle layer is located between an outer layer and an inner layer. In some embodiments, the metal of the middle layer is configured as wire. In some embodiments, the metal wire of the middle layer is configured in spiral, coil, braid, woven, or straight pattern. In some embodiments, the metal or metal wire of the middle layer comprises nitinol or stainless steel. In some embodiments, the metal wire of the middle layer is round and has a diameter of 0.0005-0.0030 inch. In some embodiments, the metal wire of the middle layer is configured in a coil with a pitch of 0.0010-0.030 inch. In some embodiments, the metal wire of the middle layer is flat and has a thickness of 0.0005-0.0060 inch and a width of 0.001-0.030 inch. In some embodiments, the metal wire of the middle layer is configured in a braid with a “picks per inch” of length (PPI) of 50-300, including a braid wound in an “under one, over two’ pattern. In one example, the wire in the proximal portion of the third catheter 175 is flat, has a thickness of 0.0005-0.0060 inch and a width of 0.001-0.030 inch, and is configured in a braid configuration with a picks per inch of length of 50-300; and the wire in the distal portion of the third catheter 175 is round, has a diameter of 0.0005-0.003 inch, and is configured in a coil pattern with a pitch of 0.0010-0.030 inch. In some embodiments, the third catheter 175 comprises a lubricious or hydrophilic coating that is present on the inner surface, the outer surface, or both the inner and outer surface of the third catheter 175. In some embodiments, a lubricious or hydrophilic coating is present on the distal portion of the third catheter 175, but absent from the proximal portion of the third catheter 175. In some embodiments, the proximal end of the third catheter 175 comprises a material with a Shore durometer hardness of 40-90 D. In some embodiments the outer layer of the proximal end of the third catheter 175 comprises nylon. In some embodiments, the outer layer of the proximal end of the third catheter 175 comprises nylon with a Shore durometer hardness of 40-90 D. In some embodiments, the distal end of the third catheter 175 comprises a material with a Shore durometer hardness of 20-60 D. In some embodiments, the outer layer of the distal end of the third catheter 175 comprises Pebax or urethane. In some embodiments, the outer layer of the distal end of the third catheter comprises Pebax with a Shore durometer hardness of 40-90 D. In some embodiments, the distal end of the third catheter 175 comprises a marker band that is conspicuous during fluoroscopy and is configured to identify the location of the tip of the third catheter 175. In some embodiments, the internal or luminal diameter of the third catheter 175 is 0.033-0.098 inch. In some embodiments, the external diameter of the third catheter 175 is 0.039-0.114 inch. In some embodiments, the third catheter 175 comprises a proximal hub 180 and a shaft. In some embodiments, the length of the third catheter 175 is 40-235 cm. In some embodiments, the wall of the third catheter 175 is continuous from the proximal end to the distal end. In some embodiments, the wall of a distal portion of the third catheter 175 comprises openings for the fluid to pass out of the lumen 164 of the third catheter 175.

In some embodiments, a detachable balloon catheter 1 comprises a detachable balloon-like implant 10 and a catheter assembly 5. In some embodiments of a detachable balloon catheter 1 comprising a catheter assembly 5, a first lumen is defined by an annular gap between an inner surface of the first catheter 173 and an outer surface of the second catheter 174. In some embodiments of a detachable balloon catheter 1 comprising a catheter assembly 5, a fluid communication can be made between the proximal hub 179 of the first catheter 173, the first lumen 162, and the central void 115 or interior volume of the balloon-like implant. In some embodiments of a detachable balloon catheter 1 comprising a catheter assembly 5, the internal diameter of the first catheter 173 is 0.003-0.012 inch larger than the outer diameter of the second catheter 174. In some embodiments of a detachable balloon catheter 1 comprising a catheter assembly 5, a second lumen 163 is defined by the inner surface of the second catheter 174. In some embodiments of a detachable balloon catheter 1 comprising a catheter assembly 5, a fluid communication can be made between the proximal hub 178 of the second catheter 174 and space adjacent to the distal end of the second catheter 174. In some embodiments of a detachable balloon catheter 1 comprising a catheter assembly 5, a portion of the second catheter 174 is inserted through the one or more elastomeric or resilient valves. In some embodiments of a detachable balloon catheter 1 comprising a catheter assembly 5, an external surface of a portion of the second catheter 174 is in contact with the inner surface of the one or more elastomeric valves. In some embodiments of a detachable balloon catheter 1 comprising a catheter assembly 5, the one or more elastomeric valves seal against the second catheter 174. In some embodiments of a detachable balloon catheter 1 comprising a catheter assembly 5, the second catheter 174 is longer than the first catheter 173. In some embodiments of a detachable balloon catheter 1 comprising a catheter assembly 5, a second catheter 174 is longer than a first catheter 173, and a first catheter 173 is longer than a third catheter 175. In some examples of a detachable balloon catheter 1 comprising a catheter assembly 5, the wall of a first catheter 173 is continuous from the proximal end to the distal end and the wall of the second catheter 174 is continuous from the proximal end to the distal end. In some embodiments of a detachable balloon catheter 1 comprising a catheter assembly 5, the wall of the first catheter 173 is continuous and the wall of the segment of the second catheter 174 that passes through the central void 115 or interior volume of the balloon-like implant 10 comprises openings for fluid to pass out of the lumen 162 of the second catheter 174. In some embodiments of a detachable balloon catheter 1 comprising a catheter assembly 5, the proximal hub 178 of the second catheter 174 is proximal to the proximal hub 179 of the first catheter 173, a portion of the second catheter 174 passes through the lumen 162 of the first catheter 173, and the distal end of the second catheter 174 is distal to the distal end of the first catheter 173. In some embodiments of a detachable balloon catheter 1 comprising a catheter assembly 5, a portion of the second catheter 174 passes through a valve or a Tuohy-Borst adaptor 186 with a valve that is joined to the hub 179 of the first catheter 173. In some embodiments of a detachable balloon catheter 1 comprising a catheter assembly 5, an outer surface of a portion of the second catheter 174 makes contact with an inner surface of the valve. In some embodiments of a detachable balloon catheter 1 comprising a catheter assembly 5, a portion of the second catheter 174 is received within the distal neck 140 of the balloon-like implant 10. In some embodiments of a detachable balloon catheter 1 comprising a catheter assembly 5, a portion of the second catheter 174 passes through the distal neck 140 of the balloon-like implant 10. In some embodiments of a detachable balloon catheter 1 comprising a catheter assembly 5, the internal diameter of the distal neck 140 of the balloon-like implant 10 is 0.001, 0.002, 0.003, or 0.004 inch larger than the external diameter of the second catheter 174. In some embodiments, the gap can be larger than 0.004 inches. In some embodiments of a detachable balloon catheter 1 comprising a catheter assembly 5, a portion of the second catheter 174 is received within a ring structure, tubular structure, telescoping structure, catheter segment, or telescoping catheter segment 185 joined to the distal balloon-like implant neck 140. In some embodiments of a detachable balloon catheter 1 comprising a catheter assembly 5, a portion of the second catheter 174 is inserted through a ring structure, tubular structure, telescoping structure, catheter segment, or telescoping catheter 185 joined to the distal balloon-like implant neck 140. In some embodiments of a detachable balloon catheter 1 comprising a catheter assembly 5, the internal diameter of the ring structure, tubular structure, telescoping structure, catheter segment, or telescoping catheter segment 185 joined to the distal neck 140 of the balloon-like implant 10 is 0.001-0.004 inch larger than the external diameter of the second catheter 174. In some embodiments of a detachable balloon catheter 1 comprising a catheter assembly 5, the length of the ring structure, tubular structure, telescoping structure, catheter segment, or telescoping catheter segment joined to the distal neck 140 of the balloon-like implant 10 is 0.3-6.0 mm.

In some embodiments of a detachable balloon catheter 1 comprising a catheter assembly 5, the proximal hub of the third catheter 175 can be joined to the proximal hub 179 of the first catheter 173. In some embodiments of a detachable balloon catheter 1 comprising a catheter assembly 5, the engagement or joining of the proximal hubs 179 & 180 of the first and third catheters 173 & 175 prevents or reduces leaking during injection of fluid into the third lumen 164. In some embodiments of a detachable balloon catheter 1 comprising a catheter assembly 5, once joined, the proximal hub 180 of the third catheter 175 and the proximal hub 179 of the first catheter 173 can be separated or can be separated after rotating a lock. In some embodiments of a detachable balloon catheter 1 comprising a catheter assembly 5, the third catheter 175 can be moved forward or backward while the first catheter 173 remains fixed in position. In some embodiments of a detachable balloon catheter 1 comprising a catheter assembly 5, when the proximal hub 180 of the third catheter 175 and the proximal hub 179 of the first catheter 173 are separated, the third catheter 175 can be moved forward or backward while the first catheter 173 remains fixed in position. In some embodiments of a detachable balloon catheter 1 comprising a catheter assembly 5, when the proximal hub 180 of the third catheter 175 and the proximal hub 179 of the first catheter 173 are separated, the proximal hub 180 of the third catheter 175 comprises a valve to prevent leaking during injection of fluids into the third lumen. In some embodiments of a detachable balloon catheter 1 comprising a catheter assembly 5, the proximal hub 180 of the third catheter 175 comprises a Tuohy-Borst adaptor 186 with a valve. In some embodiments of a detachable balloon catheter 1 comprising a catheter assembly 5, the first portions of the first and second catheters 173 & 174 pass through the valve or Tuohy-Borst adaptor 186 of the third catheter 175. In some embodiments of a detachable balloon catheter 1 comprising a catheter assembly 5, the external surface of a portion of the first catheter 173 makes contact with an inner surface of the valve. In some embodiments of a detachable balloon catheter 1 comprising a catheter assembly 5, the proximal hub 179 of the first catheter 173 and the proximal hub 178 of the second catheter 174 are proximal to the proximal hub 180 of the third catheter 175. In some embodiments of a detachable balloon catheter 1 comprising a catheter assembly 5, portions of the first and second catheters 173 & 174 pass through the proximal hub 180 of the third catheter 175. In some embodiments of a detachable balloon catheter 1 comprising a catheter assembly 5, portions of the first and second catheters 173 & 174 pass through the lumen 164 of the third catheter 175. In some embodiments of a detachable balloon catheter 1 comprising a catheter assembly 5, portions of the first and second catheters 173 & 174 are distal to the distal end of the third catheter 175. In some embodiments, the proximal hub 180 of the third catheter 175 of a detachable balloon catheter 1 comprises a port 177 for the injection of fluids into the third lumen 164.

### Attachment/Detachment Systems

The present disclosure relates to medical devices 1 comprising a detachable balloon-like implant 10 and a catheter or catheter assembly 5, wherein the detachable balloon-like implants 10 are configured for detachment from the catheter or catheter assembly 5 in vivo. In some embodiments, a distal end of the first catheter assembly and a portion of a proximal neck assembly of the balloon-like implant combine to form a mechanical attachment 500 between the first catheter 173 and the balloon-like implant 10, as shown in FIGS. 11A-E. The mechanical attachment is configured to be engaged when the second catheter 174 passes through the attachment site, as shown in FIGS. 11A-C, and is configured to be disengaged when the second catheter 174 is removed from the attachment site, as shown in FIGS. 11D-E.

In some embodiments, the male tubular structure's 510 outer surface is encapsulated in the outer surface of the first catheter 173 such that when the balloon-like implant 10 is assembled onto the first catheter 173, the outer surface of the first catheter 173 forms a gasket type seal against either the female structure 520, the proximal neck assembly, or both.

In some embodiments, the detachable balloon-like implant 10 is operably coupled and decoupled from the first catheter 173 by the opening and closing of a mechanical latch 500, as shown in FIGS. 11A-E. The mechanical latch 500 comprises a tubular male structure 510 assembled to the distal end of the first catheter 173. The mechanical latch 500 further comprises a tubular female structure 520 bonded to a proximal neck 130 or proximal neck assembly of the balloon-like implant.

The male tubular structure can also have two arms and tabs, three arms and tabs, four arms and tabs, five arms and tabs, or six arms and tabs. The second catheter 174 of the detachable balloon catheter 1 is configured such that the tubular male structure 510 can be fixed to the tubular female structure 520 in one configuration, and wherein, in a second configuration, the tubular male structure 510 is free to move relative to the tubular female structure 520. When the tubular male structure 510 is received within the second detachable assembly lumen of the tubular female structure 520 and a portion of the shaft of the second catheter 174 is received within the first detachable assembly lumen of the male structure, the second catheter 174 exerts a radially outward force on the at least one arm of the tubular male structure 510 resulting in an engaged configuration wherein the tubular male structure 510 is joined to the tubular female structure. 520. When the tubular male structure 510 is received within the second detachable assembly lumen of the tubular female structure 520 and the shaft of the second catheter 174 is withdrawn from within the first detachable assembly lumen of the male structure, the detachable assembly changes from an engaged to a disengaged configuration and the assembly of the first catheter 173 and the tubular male structure 510 can be separated from the assembly of the tubular female structure 520 and the proximal neck 130 of the balloon-like implant, and the first catheter 173 and the balloon-like implant 10 can be pulled apart. In some embodiments, the internal diameter of the female tubular structure 520 is 0.0005, 0.001, 0.0015, 0.002, or 0.003 inch larger than the external diameter of the male tubular structure 510, and the male tubular structure and female tubular structure 510 & 520 are capable of engaging in a slip-fit engagement. In some embodiments, the male tubular structure 510 and female tubular structure 520 are made by machining, casting, or other suitable methods. In some embodiments, the male tubular structure 510 comprises metal, radiopaque metal, nitinol, stainless steel, platinum, iridium, gold, silver, titanium, or combinations or alloys thereof. In some embodiments, the female tubular structure 520 comprises a metal, a radiopaque metal, platinum, iridium, gold, silver, stainless steel, nitinol, titanium, cobalt chrome, or alloys or combinations thereof. When the second catheter 174 is withdrawn from within the first detachable assembly lumen of the male structure and the detachable assembly changes from an engaged to a disengaged configuration, the assembly of the first catheter and the tubular male structure 510 can be separated from the assembly of the proximal neck 130 of the balloon-like implant 10 and the tubular female structure 520 by pulling the first catheter 173 and the balloon-like implant 10 apart.

In some embodiments, internal or luminal diameter of the tubular male structure 510 is 0.025-0.068 inch. In some embodiments, the external diameter of the tubular male structure 510 is 0.028-0.097 inch. In some embodiments, the internal and the external diameter of the tubular female structure 520 is 0.031-0.096 inch.

In one embodiment, the attachment and detachment assembly for a detachable balloon catheter 1 includes mated parts. The male tubular structure is received within the female tubular structure. The male tubular structure is a generally tubular structure having a wall that defines a lumen or conduit. The lumen extends from a proximal end of the male tubular structure 510 to the distal end. The lumen is dimensioned to receive the mobile second catheter 174 while also providing a conduit for fluid through the detachment assembly for inflation of the detachable balloon-like implant. The male and female tubular structures are machined with close tolerances to engage each other in a slip-fit engagement.

The wall also includes one or more depth stop projections that engage complimentary recesses in the proximal face of the female tubular structure. In addition to limiting insertion of the male tubular structure into the female tubular structure, depth stop projections also permit rotation of the female tubular structure and detachable balloon-like implant, when the delivery device is rotated. In various embodiments, the male tubular structure is constructed of nitinol or stainless steel. When the mechanical latch is mated, the annular shoulder of the male component contacts the annular shoulder of the female component, among others, allowing the transmission of axial compressive force from the first catheter 173 to the proximal neck 130 of the detachable balloon-like implant 10.

The female tubular structure 520 includes a tubular wall defining a lumen that is dimensioned to receive the male tubular structure. The proximal end of the female tubular structure includes an annular flange that defines the one or more recesses. The distal end of the female tubular structure is bonded to the proximal neck 130 of the detachable balloon-like implant. In various embodiments, the female tubular structure is composed of a radiopaque metal including but not limited to platinum, rhodium, or alloys thereof.

To allow detachment of the detachable balloon-like implant 10 from the first catheter 173, the second catheter 174 is retracted from the first catheter 173. The first catheter 173 is then retracted, un-mating the mechanical latch 500. It should be noted that the detachment process can occur with the guidewire 40 in place. It should also be noted that the detachment process can occur with a coil or other elongated or expandable body 720 in place of the guidewire 40. The mechanical latch 500 can be used for the attachment of detachable balloon-like implants 10 and other expandable bodies of various sizes and shapes to catheter systems.

The mechanical latch 500 is compatible with deployment of the detachable balloon-like implant 10 when it is used alone, as shown in FIGS. 11A-E; used in combination with one or more elongated bodies or expandable bodies 720 placed within the balloon-like implant 10; used in combination with one or more braid components 300 and/or balloon-like implants 200; or used in combination with one or more elongated bodies or expandable bodies 720 placed both within and distal to the balloon-like implant 10.

FIGS. 11A-E show a first sequence of operation of a detachable balloon catheter 1 with a mechanical latch 500 attachment system according to one embodiment. The balloon-like implant 10 is positioned and expanded. The guidewire 40 is retracted. The second catheter 174 is retracted from the male and female tubular structures 510 & 520 of the latch to detach the first catheter 173 from the proximal neck 130 of the balloon-like implant 10. Finally, the first catheter 173 is retracted. In some embodiments, before balloon-like implant 10 is positioned and expanded, a braid component 300 can be positioned and deployed. A distal anchor portion 301 of braid component 300 can be positioned and/or deployed outside of the balloon-like implant 10 and a proximal support portion 302 can be positioned and/or inside of balloon-like implant 10. In some embodiments, guidewire 40 can run through a side catheter 602 and out of an opening 608, where guidewire 40 is outside of the delivery catheter, along a side of the balloon-like implant. Guidewire 40 can interact with the braid component 300, such as with leading loop 402, to guide and position the braid component and/or balloon-like implant 10 as part of a dual delivery system 600. In this regard, the balloon-like implant 10 and braid component 300 can be delivered to a desired location at the same time.

In some embodiments, when an expanded detachable balloon-like implant 10 of a detachable balloon catheter 1 comprising one or more elastomeric or resilient valves joined to a distal neck 140 or distal neck assembly of the detachable balloon-like implant 10 is expanded in an artery 317, vein 318, LAA, aneurysm, biological conduit, or other blood containing space or biological space 904 and at least a portion of the external surface of the balloon-like implant 10 or a portion of the external surface of an expandable retention structure 731 attached to the balloon-like implant 10 is in contact with at least a portion of the wall of the artery 317, vein 318, LAA, aneurysm, biological conduit, or other blood containing space or biological space 904, the second catheter 174 can be separated from the expanded detachable balloon-like implant 10 by pulling the second catheter 174 and the expanded detachable balloon-like implant 10 apart.

In some examples, when the detachable balloon-like implant 10 of a detachable balloon catheter 1 is expanded in an artery 317, vein 318, LAA, aneurysm, biological conduit, or other blood containing space or biological space 904 and at least a portion of the external surface of the expanded detachable balloon-like implant 10 or a portion of the external surface of an expandable retention structure 731 attached to the balloon-like implant 10 is in contact with at least a portion of the wall of the artery 317, vein 318, LAA, aneurysm, biological conduit, or other blood containing space or biological space 904, the second catheter 174 can be separated from the expanded detachable balloon-like implant 10 by pulling the second catheter 174 and the expanded balloon-like implant 10 apart.

In some examples, when the detachable balloon-like implant 10 of a detachable balloon catheter 1 is expanded in an artery 317, vein 318, LAA, aneurysm, biological conduit, or other blood containing space or biological space 904 and at least a portion of the external surface of the expanded detachable balloon-like implant 10 or a portion of the external surface of an expandable retention structure 731 attached to the balloon-like implant 10 is in contact with at least a portion of the wall of the artery 317, vein 318, LAA, aneurysm, biological conduit, or other blood containing space or biological space 904, an assembly of the second catheter 174 and the first catheter 173 can be separated from the expanded detachable balloon-like implant 10 by pulling the assembly of the second catheter 174, the first catheter 173, and the expanded balloon-like implant 10 apart.

In some examples, one or more elastomeric or resilient valves are configured to close a distal opening, distal neck 140, distal telescoping segment 185, or distal neck assembly of the expanded detachable balloon-like implant 10 when the second catheter 174 is separated from the expanded detachable balloon-like implant 10. In some examples, one or more elastomer or resilient valves are configured to reduce blood flow through the central void 115 or interior volume of the expanded detachable balloon-like implant 10 following removal of the second catheter 174 from the expanded detachable balloon-like implant 10.

In some embodiments, the external diameter of one or more elastomeric or resilient valves is 0.018-0.068 inch. In some embodiments, the external diameter of one or more elastomeric or resilient valves is larger than 0.068 inch. It should be noted that, with a valve configured for a friction fit, the detachment process can occur either with the guidewire 40 extending through the valve and terminating distal to the expanded detachable balloon-like implant 10, or without the guidewire 40 present. It should also be noted that the detachment process can occur with a coil or other elongated or expandable body 720 in the lumen 162 of the first catheter 173 or in the lumen 163 of the second catheter 174.

### Complete Detachable Balloon Catheters or First Medical Devices

A medical device 1 comprising a detachable balloon-like implant can further comprise a first catheter 173 that is coupled to the proximal end of the detachable balloon-like implant 10. In some embodiments, the first catheter is coupled to a proximal neck 130 of the detachable balloon-like implant 10. In some embodiments, the first catheter 173 is coupled to the detachable balloon-like implant 10 by a friction fit. In some embodiments, the first catheter 173 is coupled to the detachable balloon-like implant 10 by a glue, adhesive, solder, or weld.

A medical device 1 comprising a detachable balloon-like implant can further comprise a second catheter 174 that passes through the central void 115 of the detachable balloon-like implant and couples with the proximal end or proximal neck 140 of the detachable balloon-like implant. In some embodiments, the tip of the second catheter 174 further comprises one or two marker bands that are conspicuous during fluoroscopy and configured to assist in the delivery and detachment of first expandable bodies 720 from second expandable bodies.

In some embodiments of a medical device comprising a detachable balloon-like implant 10, the tip of the second catheter 174 can be rendered mobile and advanced into the biological space 904 adjacent or distal to the expanded detachable balloon-like implant 10. As used herein, rendering the second catheter 174 mobile can refer to manipulating a control mechanism on its proximal hub 178, or turning the hemostatic valve of a Tuohy Borst adaptor 186 on a first catheter 173 to reduce friction between the hemostatic valve and the external surface of the adjacent second catheter 174, such that the second catheter 174 can be advanced forward into the biological space 904 distal to the remaining fixed assembly of the detachable balloon-like implant 10 and the first catheter 173, and steered or guided to a desired location. The second catheter 174 can also be rendered mobile and navigable by any other suitable means including but not limited to mechanical arrangements, magnetic interaction, or the use of an electrical current, or combinations thereof, among others.

In some embodiments of a medical device 1 comprising a detachable balloon-like implant 10, the tip of the second catheter 174 can be advanced into the biological space distal to the detachable balloon-like implant 10 when expanded. One or more first elongated bodies 720 or expandable bodies can be placed through the lumen 163 of the second catheter 174 and into the biological space 904 adjacent to the expanded balloon-like implant 10 prior to detachment of the first catheter 173 from the detachable balloon-like implant 10. When used in this way, the second catheter 174 can also be referred to as a “coiling catheter.” In some embodiments of a medical device 1 comprising a detachable balloon-like implant 10, the tip of the second catheter 174 can be pulled back into the central void 115 of the detachable balloon-like implant 10 when expanded. One or more first elongated bodies 720 or expandable bodies can be placed through the lumen 163 of the second catheter 174 and into the central void 115 of the detachable balloon-like implant 10 prior to detachment of the first catheter 173 from the detachable balloon-like implant 10. One or more first elongated bodies or expandable bodies 720 can be placed through the lumen 163 of the second catheter 174 and into the remaining portion of the lumen 322 of the aneurysm that is not filled with the expanded balloon-like implant 10. The tip of the second catheter 174 can be repositioned in the remaining portion of the lumen of the aneurysm that is not filled with the expanded balloon-like implant 10 and then one or more additional first elongated bodies or expandable bodies 720 or medical devices can be placed. The tip of the second catheter 174 can be repositioned again in the remaining portion of the lumen of the aneurysm that is not filled with the expanded balloon-like implant 10 and then one or more additional first elongated bodies or expandable bodies 720 or medical devices can be placed. The assembly of the first and second catheters 173 & 174 can be separated or detached from the expanded balloon-like implant 10 and the first and second catheters 173 & 174 can be removed. Manipulation of components at the hub can cause the advancement or retraction of the second catheter 174 relative to the expanded balloon-like implant 10, first catheter 173, or third catheter 175. The second catheter 174 can be rendered mobile and advanced forward relative to the expanded balloon-like implant 10, first catheter 173, or third catheter 175 to facilitate the placement of one or more first elongated bodies or expandable bodies 720 or medical devices in the unfilled lumen of the aneurysm behind the expanded balloon-like implant 10. The second catheter 174 can further comprise one or more radiopaque marker bands to help identify the catheter tip position using fluoroscopy, helping facilitate the placement and detachment of the first elongated bodies or expandable bodies 720 or medical devices that are placed through the lumen 163 of the second catheter 174.

In some embodiments, a ring structure, tubular structure, telescoping structure, catheter segment, or telescoping catheter segment 185 is bonded to the distal neck 140 of the detachable balloon-like implant and helps to form a tight seal between the outer surface of the second catheter 174 and the distal neck 140 or neck assembly of the detachable balloon-like implant 10 to facilitate expansion of the detachable balloon-like implant 10 or to facilitate the sliding of the distal neck 140 of the detachable balloon-like implant 10 on the second catheter 174 during expansion of the detachable balloon-like implant 10. During expansion of the detachable balloon-like implant 10, the ring structure, tubular structure, telescoping structure, catheter segment, or telescoping catheter structure 185 allows the body of the detachable balloon-like implant 10 to shorten in the axial direction 706. For a medical device 1 comprising a detachable balloon-like implant 10 intended for use in the treatment of saccular aneurysms, this maximizes the distance between the distal end of the expanded detachable balloon-like implant 10 and the dome of the aneurysm so that a first elongated body 720 or an expandable body can be placed in the sac or lumen of the aneurysm distal to the expanded detachable balloon-like implant with the least risk of injuring, rupturing, or puncturing the often fragile dome of the aneurysm. The ring structure, tubular structure, telescoping structure, catheter segment, or telescoping catheter segment 185 can also reduce leakage of the injected fluid medium from the detachable balloon-like implant during expansion, which reduces the pressure required for expansion and reduces the rate of collapse of detachable polymer balloon-like implants and detachable flexible polymer balloon-like implants 14 after expansion in vivo. In some embodiments, the ring structure, tubular structure, or telescoping structure 185 can be a section of metal tubing comprising gold, platinum, iridium, tantalum, or combinations or alloys thereof that can also function as a radiopaque marker that is visible under fluoroscopy. In one aspect, the radiopaque ring structure, tubular structure, or telescoping structure enhances the visibility of the detachable balloon-like implant 10 under fluoroscopic imaging.

In various embodiments, the telescoping structure 185 can be a hypotube that remains open and unobstructed upon deployment in the vascular system. By way of example and not limitation, the hypotube is dimensioned such that blood flows through the hypotube at a rate of 7.3 ml/min or less under normal physiological conditions. In some embodiments, the rate can be 23 ml/min or less. As such, the hypotube induces thrombosis within the balloon-like implant 10 due to the slowed rate of blood flow through the balloon-like implant 10.

In another aspect, the radiopaque ring structure, tubular structure, or telescoping structure 185 can help a physician in positioning the tip of the second catheter 174 prior to the placement of all or a portion of a first elongated body 720 or expandable body in vivo, including placement within the central void 115 of, or adjacent to, an expanded detachable balloon-like implant 10. Alternatively, the ring structure, tubular structure, telescoping structure, catheter segment, or telescoping catheter segment 185 can be composed of a polymer and include a radiopaque marker spot or band to enhance the visibility of the detachable balloon-like implant 10 under fluoroscopic imaging. In another aspect, the radiopaque marker spot or band or the ring structure, tubular structure, or telescoping structure 185 can help a physician in positioning the tip of the second catheter 174 prior to the placement of all or a portion of a first elongated body 720 or expandable body 10 in vivo, including placement within the central void 115 of, or adjacent to, an expanded detachable balloon-like implant 10.

### Manufacturing of Detachable Balloon Catheters-Fabricating Detachable Polymer Balloon-Like Implants

The central layer of the wall 30 of a detachable balloon-like implant, the interior layer of the wall 30 of a detachable balloon-like implant, and an exterior layer of the wall 30 of the detachable balloon-like implant can be formed by any suitable method. In some embodiments, the wall 30 of the detachable balloon-like implant has two layers. The inner layer of the wall 30 of the detachable balloon-like implant comprises non-compliant material, nylon, or Pebax and is formed by blow molding fabrication. In some embodiments, the detachable balloon-like implant has three layers. The inner layer of the wall 30 of the detachable balloon-like implant comprises non-compliant material, nylon, or Pebax and is formed by blow molding fabrication. The central layer of the wall 30 of the detachable balloon-like implant comprises gold or titanium formed by sputter deposition or vapor deposition. The outer layer of the wall 30 of the detachable balloon-like implant comprises gold or platinum formed by electroforming or electroplating.

In some embodiments, the wires, strands, coils, coiled wires, assemblies of wires, assemblies of strands, assemblies of coils, assemblies of coiled wires, woven assemblies of wires, woven assemblies of strands, woven assemblies of coils, woven assemblies of coiled wires, braided assemblies of wires, braided assemblies of strands, braided assemblies of coils, braided assemblies of coiled wires, and combinations thereof, of expandable bodies are self-expanding. In some embodiments, the wires, strands, coils, coiled wires, assemblies of wires, assemblies of coils, assemblies of coiled wires, woven assemblies of wires, woven assemblies of coils, woven assemblies of coiled wires, braided assemblies of wires, braided assemblies of coils, braided assemblies of coiled wires and combinations thereof, of expandable bodies comprise nitinol. It should be noted that in some embodiments, other devices, structures, or medical devices can be used to fill the cavity of the balloon-like implant, such as glue or balls, that are not self-expanding.

In some embodiments, the expandable bodies can include regions, such as pitch region 408, where the pitch of the wires varies. In these regions, the wires can be wound, woven, or otherwise formed in a tighter configuration or looser configuration than the surrounding regions. The varying pitch regions allow the expandable body 720 to vary the stiffness of its structure. In some embodiments, the varying pitch regions have varying overall diameters of the region. These varying pitch and diameter regions can prevent the expandable body 720 from collapsing in on itself due to natural forces present in woven, coiled, or otherwise symmetric structure of a non-varied pitch expandable body. The variable outer diameter can allow for an accordion effect, where wire rings overlap each other, creating compaction zones thereby increasing the radial force of the fully deployed expandable body 720. For example, an expandable body, such as braid component 300, can have 5 pitch regions including a distal end region, a distal region, and intermediate region, a proximal region, and a proximal end region. In this example, the distal end region has a pitch of 2-3 mm, the distal region has a pitch of 1 mm, the intermediate region has a pitch of 1 mm, the proximal region has a pitch of 0.7 mm, and the proximal end region has a pitch of 1 mm. It should be noted that an expandable body can have 1, 2, 3, 4, 5, 6, or more varied pitch regions, can have no varied pitch regions, or any combination thereof in various possible embodiments. Although specific dimensions of varied pitch regions are discussed herein, the pitch region dimensions, such as 1 mm, can be any suitable pitch (e.g., 0.5 mm, 1 mm, 1.5 mm, 2 mm, 2.5 mm, 3 mm, 4 mm, etc.) or fraction thereof in various possible embodiments.

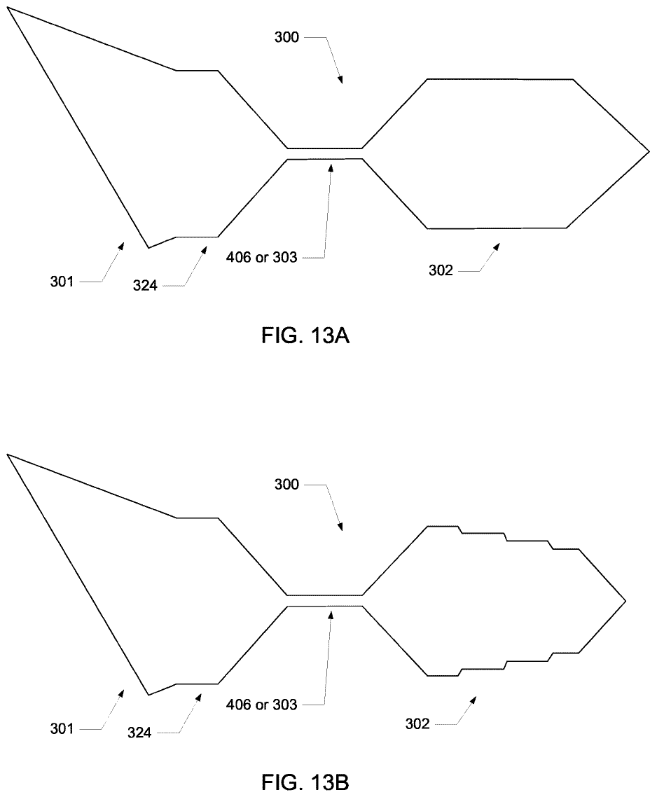

Scaled pitches across different sizes of the entire braid component and/or the balloon-like implant filling proximal portion of the braid component are possible. For example, the distal anchor portion can keep a pitch between 1-7 mm to help with expansion once unsheathed. One pitch pattern is alternating high density and low density. In one embodiment, the pitch pattern includes a tapering pattern, such as cone taper 414 shown in FIG. 4E, with decreasing outer diameters and pitches. In some embodiments, the pitch range can be from 0.1 mm to 0.5 mm. Cone tapering can allow the braid to accommodate a large vessel diameter treatment range by facilitating telescoping and compaction. This can be possible as the radial force drops off logarithmically as it approaches its heat set outer diameter. These small differences in outer diameter reduction zones, and therefore differences in radial forces (stepped reduction or linear taper), allow for wires to slip under each other over a set length proportional to the balloon-like implant length. In some embodiments, a first pitch and second pitch can be derived by the linear slope of a min and max pitch at the min and max balloon-like implant sizes. As described, the function is to reduce the collapsed braid length. The collapsed braid length can be a function of the braid circumference, pitch, and length. It should be noted that braid length divided by pitch can give the number of loops. The number of loops multiplied by the circumference would equal a straightened length approximation. For example, braid outer diameter multiplied by pi can equate to the collapsed braid length. Shapes for additional embodiments of the expandable body 300 are shown in FIGS. 13A-B. As shown, the expandable bodies 300 can include a distal anchor portion 301, that is substantially similar to the distal anchor portion shown in FIGS. 4A-E. In some embodiments, as shown in FIG. 13A the support portion may have a generally oblong shape. In another embodiment, as shown in FIG. 13B, the support portion may have a generally tapered or stepped shape. Additionally, the anchor portion 301 has a reduced length as compared to other embodiments. In particular, the anchor portion 301 includes no cylindrical portion or has a reduced length for the cylindrical portion 324 as compared, such as that found in the embodiment shown in FIG. 5B.

In some embodiments, the first elongated or expandable body of the second medical device 1 comprises a coiled wire, wherein the primary diameter of the coiled wire is 0.00175-0.003 inch (approximately 0.04 mm to 0.08 mm) in diameter, and the secondary diameter of the coiled wire is 0.010-0.050 inch (approximately 0.25 mm to 1.3 mm) in diameter. In some embodiments, the first elongated or expandable body has a tertiary structure without pre-formed loops or shapes, is configured to form a straight or unformed tertiary shape when relaxed or is configured as a straight vascular coil. In some embodiments, at least a portion of the first elongated or expandable body has a helical, spherical, or complex tertiary structure. In some embodiments, at least a portion of the first elongated or expandable body is configured to form a coiled, helical, or complex tertiary shape when relaxed. In some embodiments, the coiled wire is a vascular coil. In some embodiments, the distal portion of the first elongated or expandable body comprises one loop of tertiary structure and the remainder of the first elongated or expandable body comprises a tertiary structure without pre-formed loops or shapes when relaxed; or the distal portion of the first elongated or expandable body comprises one loop of tertiary structure and the remainder of the first elongated or expandable body comprises a tertiary structure configured to form a straight or unformed tertiary shape when relaxed. In some embodiments, the distal portion of the first elongated or expandable body comprises two loops of tertiary structure and the remainder of the first elongated or expandable body comprises a tertiary structure without pre-formed loops or shapes when relaxed, or the distal portion of the first elongated or expandable body comprises two loops of tertiary structure and the remainder of the first elongated or expandable body comprises a tertiary structure configured to form a straight or unformed tertiary shape, when relaxed. In some embodiments, the distal portion of the first elongated or expandable body comprises three loops of tertiary structure and the remainder of the first elongated or expandable body comprises a tertiary structure without pre-formed loops or shapes when relaxed, or the first elongated or expandable body comprises three loops of tertiary structure and the remainder of the first elongated or the expandable body comprises a tertiary structure configured to form a straight or unformed tertiary shape, when relaxed. In some embodiments, the distal portion of the first elongated or expandable body comprises four or more loops of tertiary structure when relaxed and the remainder of the first elongated or expandable body comprises a tertiary structure without pre-formed loops or shapes when relaxed, or the first elongated or expandable body comprises four loops of tertiary structure and the remainder of the first elongated or the expandable body comprises a tertiary structure configured to form a straight or unformed tertiary shape, when relaxed. In some embodiments, as many as 10 loops can be used. The tertiary diameter of the looped, coiled, formed, or tertiary portion of the first elongated or expandable body can be 2-100 mm. In some embodiments, the first elongated or expandable body comprises platinum, iridium, nickel, tungsten or combinations thereof.

In some embodiments, the first elongated or expandable body of the second medical device 1 is a wire with a primary diameter of 0.005-0.050 inch. In some embodiments, the first elongated or expandable body is a wire without a secondary or tertiary diameter or shape. In some embodiments, the first elongated or expandable body is a wire and without pre-formed loops or shapes. In some embodiments, the first elongated or expandable body is a wire that is configured to form a straight or unformed tertiary shape when relaxed. In some embodiments, the first elongated or expandable body is a straight vascular coil. In some embodiments, the first elongated or expandable body is a wire and at least a portion of the first elongated or expandable body has a helical, spherical, or complex tertiary structure. In some embodiments, the first elongated or expandable body is a wire, has a portion of the first elongated or expandable body has a helical, spherical, or complex tertiary structure, with the remainder of the first elongated or expandable body comprises a tertiary structure without pre-formed loops or shapes, when relaxed. In some embodiments, the first elongated or expandable body is a wire wherein the distal portion of the first elongated or expandable body comprises one loop of tertiary structure and the remainder of the first elongated or expandable body comprises a tertiary structure configured to form a straight or unformed tertiary shape when relaxed, or the distal portion of the first elongated or expandable body comprises one loop of tertiary structure and the remainder of the first elongated or expandable body comprises a tertiary structure without pre-formed loops or shapes, when relaxed. In some embodiments, the first elongated or expandable body is a wire, wherein the distal portion of the first elongated or expandable body comprises two loops of tertiary structure and the remainder of the first elongated or expandable body comprises a tertiary structure without pre-formed loops or shapes when relaxed, or the distal portion of the first elongated or expandable body comprises two loops of tertiary structure and the remainder of the first elongated or expandable body comprises a tertiary structure configured to form a straight or unformed tertiary shape, when relaxed. In some embodiments, the first elongated or expandable body is a wire, wherein the distal portion of the first elongated or expandable body comprises three loops of tertiary structure and the remainder of the first elongated or expandable body comprises a tertiary structure without pre-formed loops or shapes when relaxed, or the distal portion of the first elongated or expandable body comprises three loops of tertiary structure and the remainder of the first elongated or expandable body comprises a tertiary structure configured to form a straight or unformed tertiary shape, when relaxed. In some embodiments, the first elongated or expandable body is a wire, wherein the distal portion of the first elongated or expandable body comprises a tertiary structure configured to form a straight or unformed tertiary shape when relaxed, or the distal portion of the first elongated or expandable body comprises one loop of tertiary structure and the remainder of the first elongated or expandable body comprises a tertiary structure without pre-formed loops or shapes, when relaxed. In some embodiments, the first elongated or expandable body is a wire, wherein the distal portion of the first elongated or expandable body comprises two loops of tertiary structure and the remainder of the first elongated or expandable body comprises a tertiary structure without pre-formed loops or shapes when relaxed, or the distal portion of the first elongated or expandable body comprises two loops of tertiary structure and the remainder of the first elongated or expandable body comprises a tertiary structure configured to form a straight or unformed tertiary shape, when relaxed. In some embodiments, the first elongated or expandable body is a wire, wherein the distal portion of the first elongated or expandable body comprises three loops of tertiary structure and the remainder of the first elongated or expandable body comprises a tertiary structure without pre-formed loops or shapes when relaxed, or the distal portion of the first elongated or expandable body comprises three loops of tertiary structure and the remainder of the first elongated or expandable body comprises a tertiary structure configured to form a straight or unformed tertiary shape, when relaxed. In some embodiments, the first elongated or expandable body is a wire, wherein the distal portion of the first elongated or expandable body comprises four or more loops of tertiary structure, and the remainder of the first elongated or expandable body comprises a tertiary structure without pre-formed loops or shapes when relaxed, or the distal portion of the first elongated or expandable body comprises four loops of tertiary structure and the remainder of the first elongated or expandable body comprises a tertiary structure configured to form a straight or unformed tertiary shape, when relaxed. In some embodiments, the first elongated or expandable body is a wire and the tertiary diameter of the looped, coiled, or formed portion of the first elongated or expandable body is 2-100 mm. In some embodiments, the first elongated or expandable body is a wire comprising nitinol, a nitinol wire is plated or coated with platinum or gold, or a nitinol wire further with one or more radiopaque markers that are visible during fluoroscopy, including wherein the radiopaque marker comprises platinum, iridium, gold, tungsten, or combinations thereof, or is a radiopaque marker in the form of a ring or band around a portion of the wire.

In some embodiments, the second medical device 1 comprises a first elongated or expandable body that is configured to be carried through the lumen 163 of the second catheter 174 of the detachable balloon catheter 1 by the catheter of the second medical device 700. In some embodiments, the catheter of the second medical device comprises a radiopaque marker band that is visible during fluoroscopy, including wherein the radiopaque marker comprises platinum, iridium, gold, tungsten, or combinations thereof.

In some embodiments, the first elongated body 720 of the second medical device 720 comprises a wire assembly, coiled wire assembly, braided wire assembly, woven wire assembly, or other expandable body 720, such as an open cell laser cut structure. In some embodiments, the expandable body 720 of the second medical device comprises a wire assembly, coiled wire assembly, braided wire assembly, woven wire assembly, or other expandable body 720. In some embodiments, the first expandable body 720 of the second medical device comprises a self-expanding wire assembly, coiled wire assembly, braided wire assembly, woven wire assembly, or other expandable body 720. In some embodiments, the wire assembly, coiled wire assembly, braided wire assembly, or woven wire assembly expandable body 720 is configured to form into the shape of a generally cylindrical form when not in a compressed, collapsed, constrained, or elongated form. In some embodiments, the wire assembly, coiled wire assembly, braided wire assembly, or woven wire assembly expandable body 720 is configured to form into the shape of a generally spherical shape when not in a compressed, collapsed, constrained, or elongated form. In some embodiments, the wire assembly, coiled wire assembly, braided wire assembly, or woven wire assembly expandable body 720 is configured to form into a general shape and size with a largest diameter of 2-100 mm when not in a compressed, collapsed, constrained, or elongated form. In some embodiments, the expandable body 720 of the second medical device comprises nitinol, a nitinol wire, or a nitinol wire that is plated or coated with platinum or gold. In some embodiments, the nitinol wire, expandable body 720 further comprises one or more radiopaque markers that are visible during fluoroscopy, including radiopaque marker comprising platinum, iridium, gold, tungsten, or combinations thereof.

In some embodiments, the first elongated or expandable body of the second medical device 1 comprises a polymer strand or a polymer strand plated or coated with platinum or gold. In some embodiments, the polymer strand portion of the first elongated or expandable body of the second medical device 1 further comprises one or more radiopaque markers that are visible during fluoroscopy, including radiopaque marker comprised of platinum, iridium, gold, tungsten, or combinations thereof, and including radiopaque markers in the form of a ring or band around a portion of the polymer strand.

In some embodiments, the first elongated or expandable body is 10-400 cm in length, 70-400 cm, or 10-70 cm in length. In some embodiments, the first elongated or expandable body comprises a lubricious or hydrophilic layer or coating that is durable. In some embodiments, the first elongated or expandable body comprises a lubricious outer layer, a PTFE outer layer, a polyimide outer layer, or an outer layer comprising a PTFE and polyimide composite. In some embodiments, the second elongated body of the second medical device 1 comprises a lubricious or hydrophilic layer or coating that is durable. In some embodiments, the second elongated body of the second medical device 1 comprises a PTFE outer layer, a polyimide outer layer, or an outer layer comprising a PTFE and polyimide composite. In some embodiments, the second elongated body of the second medical device 1 comprises visual or tactile markings that enable a user to determine the length of the first elongated or expandable body that has been pushed distal to the distal tip of the second catheter 174.

In some embodiments, the first elongated or expandable body of the second medical device 1 and the second elongated body of the second medical device 1 are configured to separate by mechanical means. In some embodiments, the first elongated or expandable body of the second medical device 1 and the second elongated body of the second medical device 1 are configured to separate by electrolysis or corrosion. In some embodiments, the first elongated or expandable body of the second medical device 1 and the second elongated body of the second medical device 1 are configured to separate in a region between the first elongated or expandable body and second elongated body that is sensitive to electrolysis or corrosion or are configured to separate in a region between the first elongated or expandable body and second elongated body comprising stainless steel. In some embodiments, the second elongated body of the second medical device 1 is configured to enable the passage of an electrical current from a proximal portion of the second elongated body to the region that is sensitive to electrolysis or corrosion. In some embodiments, at least a portion of the second elongated body of the second medical device 1 is configured to enable the passage of a direct electrical current. In some embodiments, at least a portion of the second elongated body of the second medical device 1 is covered with a substance that insulates it from electrical conduction. In some embodiments, at least a portion of the segment sensitive to electrolysis or corrosion, or configured for dissolution by electrolysis, is not covered with a substance that insulates it from electrical conduction. In some embodiments, the first elongated or expandable body of the second medical device 1 and the second elongated body of the second medical device 1 are configured to separate by an electrothermal process. In some embodiments, the separation occurs in a region between the first elongated or expandable body and the second elongated body can melt with heating. In some embodiments, the second medical device 1 is configured to enable the passage of an electrical current from a proximal portion of the second elongated body to a resistive heating element on or near the region between the first elongated or expandable body and the second elongated body that can melt with heating. In some embodiments, at least a portion of the second elongated body of the second medical device 1 is covered with a substance that insulates it from electrical conduction. In some embodiments, the first elongated or expandable body of the second medical device 1 and the second elongated body of the second medical device 1 are not joined and the second elongated body of the second medical device 1 is configured to push the first elongated or expandable body of the second medical device 1 through the lumen of the second catheter 174 of the first medical device 1. In some embodiments, the second elongated body of the second medical device is configured to expel the first elongated or expandable body 720 of the second medical device from the distal end of the lumen 163 of the second catheter 174 of the first medical device 1. In some embodiments, the second elongated body of the second medical device can be removed from the lumen 163 of the second catheter 174 of the first medical device 1 after expulsion of the first elongated or expandable body 720 of the second medical device from the distal end of the lumen 163 of the second catheter 174 of the first medical device 1.

In some examples, beads, balls, microspheres, bioresorbable materials, adhesives, glues, solidifying polymers, solidifying foams, or combinations thereof are passed from the proximal end of the second catheter 174, through the lumen 163 of the second catheter 174, and into the central void 115 or interior volume of the detachable balloon-like implant 10 of a detachable balloon catheter 1 to help maintain the expanded size and shape of the detached balloon-like implant 10.

### Guidewires

The lumen 163 of the second catheter 174 can be configured to accept wires, such as guidewires, with a diameter of 0.01-0.038 inch (approximately 0.25 mm to 1 mm), including guidewires with a diameter of 0.010-0.038 inch (approximately 0.25 mm to 1 mm). The lumen 163 of the second catheter 174 can be configured to accept guidewires with a length of 50-500 cm or 200-400 cm. It should be noted that a guidewire, as used herein, can be a tube or microcatheter.

### Systems Comprising a First Medical Device and One or More Second Medical Devices

In some embodiments, the first elongated or expandable body of the second medical device 1 comprises a wire assembly, coiled wire assembly, braided wire assembly, woven wire assembly, or other expandable body. In some embodiments, the first elongated or expandable body of the second medical device 1 comprises a self-expanding wire assembly, coiled wire assembly, braided wire assembly, woven wire assembly, or other expandable body. In some embodiments, the wire assembly, coiled wire assembly, braided wire assembly, or woven wire assembly expandable body is configured to form into the general shape and size of the expanded detachable balloon-like implant of the first medical device 1 when not in a compressed, collapsed, constrained, or elongated form.

In some embodiments, the second elongated body of the second medical device comprises visual or tactile markings that enable a user to determine the length of the first elongated or expandable body 720 that has been pushed distal to the distal tip of the second catheter 174. In some embodiments, the hub 178 of the second catheter 174 of the detachable balloon catheter 1 is configured to allow for the insertion of the distal end of a first elongated body or an expandable body 720 into the lumen 163 of the second catheter 174 of the detachable balloon catheter 1, and also configured to be joined with the distal end of a carrier structure or carrier that houses, constrains, or otherwise engages the first elongated or expandable body 720 and at least a portion of the second elongated body. This helps a physician use long or very long coils, up to 400 cm or more in length by providing a longer rigid pathway to guide the coil or first elongated body 720 into the patient. In one example, the second catheter 174 of the detachable balloon catheter 1 is 130 cm in length and the carrier structure is 300 cm in length. If the hub 178 of the second catheter 174 of the detachable balloon catheter 1 is joined to the carrier, then a 400 cm first elongated or expandable body 720 can be used, with a second elongated body that is 430 cm long. Once the distal end of the second elongated body is pushed into the proximal end of the second catheter 174 of the detachable balloon catheter 1, then the carrier and the second catheter 174 of the detachable balloon catheter 1 can be separated, the second elongated body can be removed from the carrier and used to push the remainder of the first elongated or expandable body 720 out of the second catheter 174 of the detachable balloon catheter 1. In some embodiments, the carrier is configured into a coiled shape. In some embodiments, a portion of the first or expandable elongated body 720 of the second medical device 1 is configured to contact the interior surface of the expanded detachable balloon-like implant of the first medical device 1. In some embodiments, the largest overall diameter or tertiary diameter of the first elongated or expandable body 720 of the second medical device is in a range from 5% smaller than the largest diameter of the expanded detachable balloon-like implant 10 of the first medical device 1 to 20% larger than the largest diameter of the expanded detachable balloon-like implant of the first medical device 1. In some embodiments, the largest overall or tertiary diameter of the first elongated or expandable body 720 of the second medical device is 1, 2, 3, 4, 5, 6, 7, 8, 9, or 10 mm larger than the largest diameter of the expanded detachable balloon-like implant of the first medical device 1. In some embodiments, the volume of the one or more first elongated or expandable bodies of the second medical device 1 would fill 1-75% of the volume of the central void 115 of the expanded detachable balloon-like implant.

### Kits Comprising First and Second Medical Devices

Aspects and embodiments related to kits incorporating first and second medical devices as disclosed herein. Such kits comprise at least one first medical device and one or more second medical devices, configured for use with the first medical device.

In some embodiments, a kit can contain one first medical device configured for use with a guidewire with a detachable polymer balloon-like implant 10 and a second medical device wherein the expandable body 720 is a braid component when placed entirely within the central void 115 of the expanded detachable balloon-like implant 10 of the first medical device results in a support structure capable of aiding the balloon-like implant in keeping its expanded shape.

Such kits can further comprise additional medical devices. In one embodiment, a kit can comprise a first medical device, a second medical device, and a guidewire that is configured for use with the first medical device, including configured for passage through the lumen 163 of the second catheter 174 of the first medical device and with a length that is longer than the length of the second catheter 174 of the first medical device.

In some embodiments, a kit can comprise a first medical device, a second medical device, and a detachment controller that is configured to i) cause separation of the first catheter 173 and the detachable balloon-like implant 10 of the first medical device, ii) cause separation of the first expandable body 720 from the second catheter, or iii) cause separation of the first catheter 173 and the detachable balloon-like implant 10 of the first medical device 1 and also to cause separation of the first expandable body 720 from the second catheter. In some embodiments, a kit can further comprise one or more cables or connectors that make an electrical connection between i) the first medical device 1 and a controller, ii) the second medical device and a controller or iii) the first medical device 1 and a controller, and the second medical device and a controller.

In some embodiments, the kit can comprise a wire for supporting and releasably securing at least one of the balloon-like implant and the expandable device, such as braid component 300. The kit can include the balloon like-implant, one to three catheters, and a guide (e.g., a microcatheter, wire, or tube). In one embodiment, kit can also include fluid for expanding the balloon-like implant. It should be noted that all kits described herein can also include instructions for use.

### Example of Device Use in a Saccular Aneurysm

A sequence of steps is associated with the deployment of the detachable balloon-like implant in a saccular aneurysm. Initially, a guidewire can be placed so that its distal tip lies within the lumen of the aneurysm sac. Next, the first medical device comprising a pleated and folded detachable balloon-like implant can be advanced over the guidewire and through the neck or mouth of the aneurysm. After the pleated and folded detachable balloon-like implant has been placed in the lumen of the aneurysm sac, radiographic or X-ray contrast agent can be injected into the parent artery during fluoroscopy to evaluate the position of the pleated and folded detachable balloon-like implant. Once proper positioning of the detachable balloon-like implant in the lumen of the aneurysm sac has been achieved and confirmed, then the detachable balloon-like implant is inflated or expanded. A fluid medium source, such as syringe, inflation device (e.g., Endoflator® by Karl Storz, not shown) or, pump is connected to the inflation port on the hub of the first catheter and a fluid medium is injected into the central void of the detachable balloon-like implant, which results in expansion of the detachable balloon-like implant until it fills at least a portion of the lumen of the aneurysm sac. After expansion, the first catheter is pulled back in the aneurysm lumen to draw the expanded detachable balloon-like implant towards the neck of the aneurysm. After the expanded balloon-like implant has been positioned in the neck of the aneurysm, radiographic or X-ray contrast agent can be injected into the parent artery during fluoroscopy to evaluate the position of the expanded balloon-like implant and to confirm aneurysm neck occlusion. The second catheter can then be advanced into the lumen of the aneurysm sac, the guidewire removed, and one or more first elongated bodies or coils can be fed through the lumen of the second catheter (second lumen) into the lumen of the aneurysm sac. The first elongated bodies or coils exert a continuous force on the detachable balloon-like implant which provides a tight seal between the expanded balloon-like implant and the aneurysm neck. After placement of elongated bodies or coils into the lumen of the aneurysm sac, radiographic or X-ray contrast agent can be injected into the parent artery during fluoroscopy to evaluate the position of the first elongated bodies or coils and to confirm aneurysm neck occlusion. The second catheter can then be retracted into the central void of the detachable balloon-like implant and one or more first elongated bodies or coils can be fed through the lumen of the second catheter (second lumen) into the central void of the expanded detachable balloon-like implant. The first elongated bodies or coils exert an outward force on the inner wall of the expanded detachable balloon-like implant to keep the expanded detachable balloon-like implant expanded; maintain contact between the outer wall of the detachable balloon-like implant and the inner wall of the aneurysm; and prevent collapse, compression, or compaction of the detachable balloon-like implant due to external compressive forces, including external compressive forces from portions of the first elongated bodies or coils that are present in the lumen of the aneurysm sac. The first elongated bodies or coils placed within the lumen of the aneurysm sac and central void or interior volume of the detachable balloon-like implant can either be separate or the same. In some embodiments, the same first elongated bodies or coils are placed in both locations, and the distal portion of the first elongated body or coil is first placed in the lumen of the aneurysm sac and then the second catheter is retracted to allow the proximal portion of the first elongated body or coil to be placed in the central void of the detachable balloon-like implant. Finally, the first catheter is detached from the detachable balloon-like implant and removed (along with the second catheter), leaving the detachable balloon-like implant and the elongated bodies or coils within the lumen of the aneurysm sac and the central void of the detachable balloon-like implant to occlude the neck and sac the aneurysm.

### Example of Device Use in an Artery, Vein, or Biological Conduit

A sequence of steps is associated with the deployment of a detachable balloon-like implant in an artery, vein, or biological conduit. Initially, a guidewire can be placed so that its distal tip lies within the lumen of the artery, vein, or biological conduit. Next, the first medical device comprising a pleated and folded detachable balloon-like implant can be advanced over the guidewire and into a segment of the artery, vein, or biological conduit that is selected for occlusion. After the pleated and folded detachable balloon-like implant has been placed in the lumen of the selected segment of artery, vein, or biological conduit, radiographic or X-ray contrast agent can be injected into the artery, vein, or biological conduit during fluoroscopy to evaluate the position of the pleated and folded detachable balloon-like implant. Once proper positioning of the detachable balloon-like implant in the lumen of the artery, vein, or biological conduit has been achieved and confirmed, then the detachable balloon-like implant is inflated or expanded. A fluid medium source, such as syringe, inflation device (e.g., Endoflator® by Karl Storz, not shown) or, pump is connected to the inflation port on the hub of the first catheter and a fluid medium is injected into the central void of the detachable balloon-like implant, which results in expansion of the detachable balloon-like implant until it fills at least a portion of the lumen of the artery, vein, or biological conduit. After the expanded balloon-like implant has been positioned in the lumen of the artery, vein, or biological conduit, radiographic or X-ray contrast agent can be injected into the lumen of the artery, vein, or biological conduit during fluoroscopy to evaluate the position of the expanded balloon-like implant and to confirm artery, vein, or biological conduit occlusion. The guidewire can be removed. Optionally, a solution comprising a drug or therapeutic agent, a solution or suspension comprising embolic particles, or combinations thereof injected into the lumen of the target segment of artery, vein, or biological conduit. The distal end of a first elongated body or coil can be fed through the lumen of the second catheter (second lumen) into the lumen of the artery, vein, or biological conduit adjacent and distal to the expanded detachable balloon-like implant. The second catheter can then be retracted into the central void of the detachable balloon-like implant and the remained of the first elongated body or coil can be fed through the lumen of the second catheter (second lumen) into the central void of the expanded detachable balloon-like implant. Optionally, additional first elongated bodies or coils can be fed through the lumen of the second catheter (second lumen) into the central void of the expanded detachable balloon-like implant. The first elongated bodies or coils exert an outward force on the inner wall of the expanded detachable balloon-like implant to keep the expanded detachable balloon-like implant expanded; maintain contact between the outer wall of the detachable balloon-like implant and the inner wall of the artery, vein, or biological conduit; and prevent collapse, compression, or compaction of the detachable balloon-like implant due to external compressive forces, including external compressive forces from portions of the first elongated bodies or coils that are present in the lumen of the artery, vein, or biological conduit. The first elongated bodies or coils placed within the lumen of the artery, vein, or biological conduit and central void or interior volume of the detachable balloon-like implant can either be separate or the same. Finally, the first catheter (along with the second catheter) is detached from the detachable balloon-like implant leaving the detachable balloon-like implant and the elongated bodies or coils within the lumen of the artery, vein, or biological conduit and the central void of the detachable balloon-like implant to occlude the artery, vein, or biological conduit. It should be noted that the expandable body, such as braid component 300, can be deployed prior to the balloon-like implant expansion. In this regard, a portion, such as the distal anchor portion, can be deployed and expanded before the balloon-like implant is expanded.

### Example of Device Use in a Left Atrial Appendage (LAA)

A sequence of steps is associated with the deployment of the detachable balloon-like implant in a left atrial appendage (LAA). Initially, a guidewire can be placed so that its distal tip lies within the lumen of the LAA. Next, the first medical device comprising a pleated and folded detachable balloon-like implant can be advanced over the guidewire and through the neck or mouth of the LAA. After the pleated and folded detachable balloon-like implant has been placed in the lumen of the LAA sac, radiographic or X-ray contrast agent can be injected into the parent artery during fluoroscopy to evaluate the position of the pleated and folded detachable balloon-like implant. Once proper positioning of the detachable balloon-like implant in the lumen of the LAA sac has been achieved and confirmed, then the third catheter is retracted, leading to expansion of the retention structure and engagement of the arms and hooks of the retention structure in the wall of the LAA. A gentle tug on the first catheter confirms adequate wall engagement. The detachable balloon-like implant is inflated or expanded. A fluid medium source, such as syringe, inflation device (e.g., Endoflator® by Karl Storz, not shown), or pump is connected to the inflation port on the hub of the first catheter and a fluid medium is injected into the central void of the detachable balloon-like implant, which results in expansion of the detachable balloon-like implant until it fills at least a portion of the lumen of the LAA sac. After expansion of the detachable balloon-like implant, radiographic or X-ray contrast agent can be injected into the left atrium during fluoroscopy to evaluate the position of the expanded balloon-like implant and to confirm LAA occlusion. The second catheter can be retracted into the central void of the expanded detachable balloon-like implant and one or more first elongated bodies or coils can be fed through the lumen of the second catheter (second lumen) into the central void of the expanded detachable balloon-like implant. The first elongated bodies or coils exert an outward force on the inner wall of the expanded detachable balloon-like implant to keep the expanded detachable balloon-like implant expanded; maintain contact between the outer wall of the detachable balloon-like implant and the inner wall of the LAA; and prevent collapse, compression, or compaction of the detachable balloon-like implant due to external compressive forces, including external compressive forces. Finally, the first catheter is detached from the detachable balloon-like implant and removed (along with the second catheter and third catheter), leaving the detachable balloon-like implant, the retention structure, and the elongated bodies or coils within the central void of the detachable balloon-like implant to occlude the neck and sac of the LAA.

### Example of Device Use in a Left Atrial Appendage (LAA)

A sequence of steps is associated with the deployment of the detachable balloon-like implant in a LAA. Initially, a guidewire can be placed so that its distal tip lies within the lumen of the LAA sac. Next, the first medical device comprising a pleated and folded detachable balloon-like implant can be advanced over the guidewire into the lumen of the LAA and positioned such that the proximal end of the pleated and folded detachable balloon-like implant is in the neck of the LAA. After positioning of the pleated and folded detachable balloon-like implant, radiographic or X-ray contrast agent can be injected into the parent artery during fluoroscopy to evaluate the position of the pleated and folded detachable balloon-like implant. Once proper positioning of the detachable balloon-like implant in the lumen of the LAA has been achieved and confirmed, then the detachable balloon-like implant is inflated or expanded. A fluid medium source, such as syringe, inflation device (e.g., Endoflator® by Karl Storz, not shown) or, pump is connected to the inflation port on the hub of the first catheter and a fluid medium is injected into the central void of the detachable balloon-like implant, which results in expansion of the detachable balloon-like implant until it occludes the neck of the LAA and fills at least a portion of the lumen of the LAA sac. After the expanded balloon-like implant has been positioned in the neck of the LAA, radiographic or X-ray contrast agent can be injected into the parent artery during fluoroscopy to evaluate the position of the expanded balloon-like implant and to confirm LAA neck occlusion. The second catheter can then be advanced into the lumen of the LAA sac, the guidewire removed, and one or more first elongated bodies or coils can be fed through the lumen of the second catheter (second lumen) into the lumen of the LAA sac. The first elongated bodies or coils exert a continuous force on the detachable balloon-like implant which provides a tight seal between the expanded balloon-like implant and the LAA neck. After placement of elongated bodies or coils into the lumen of the LAA sac, radiographic or X-ray contrast agent can be injected into the parent artery during fluoroscopy to evaluate the position of the first elongated bodies or coils and to confirm LAA neck occlusion. The second catheter can then be retracted into the central void of the detachable balloon-like implant and one or more first elongated bodies or coils can be fed through the lumen of the second catheter (second lumen) into the central void of the expanded detachable balloon-like implant. The first elongated bodies or coils exert an outward force on the inner wall of the expanded detachable balloon-like implant to keep the expanded detachable balloon-like implant expanded; maintain contact between the outer wall of the detachable balloon-like implant and the inner wall of the LAA; and prevent collapse, compression, or compaction of the detachable balloon-like implant due to external compressive forces, including external compressive forces from portions of the first elongated bodies or coils that are present in the lumen of the LAA sac. The first elongated bodies or coils placed within the lumen of the LAA sac and central void or interior volume of the detachable balloon-like implant can either be separate or the same. In some embodiments, the same first elongated bodies or coils are placed in both locations, and the distal portion of the first elongated body or coil is first placed in the lumen of the LAA sac and then the second catheter is retracted to allow the proximal portion of the first elongated body or coil to be placed in the central void of the detachable balloon-like implant. Finally, the first catheter is detached from the detachable balloon-like implant and removed (along with the second catheter), leaving the detachable balloon-like implant and the elongated bodies or coils within the lumen of the LAA sac and the central void of the detachable balloon-like implant to occlude the neck and sac the LAA.

It will be appreciated that the devices and methods of the present disclosure are capable of being incorporated in the form of a variety of embodiments, only a few of which have been illustrated and described above. The disclosures herein can be embodied in other specific forms without departing from its spirit or essential characteristics. The described embodiments are to be considered in all respects only as illustrative and not restrictive and the scope of the present invention is, therefore indicated by the appended claims rather than by the foregoing description. All changes that come within the meaning and range of equivalency of the claims are to be embraced within their scope.

1. A medical device for placement in a biological space, the medical device comprising:
a compressed balloon-like implant configured for implantation in the biological space, the balloon-like implant comprising:
a distal region, a proximal region generally opposite the distal region, an intermediate region transitioning from the distal region to the proximal region, a first axis extending proximal-distal between the proximal region and distal region, and a second axis perpendicular to the first axis;
a wall extending generally continuously from the proximal region through the intermediate region, to the distal region, with an exterior surface and an interior surface, the interior surface defining a central void or interior volume, wherein the central void or interior volume is configured to be modified based on deployment;
an opening in the wall at a proximal end of the proximal region that allows for the passage of fluid from a first catheter into the central void or interior volume of the balloon-like implant and also allows for passage of a portion of a second catheter into the central void or interior volume of the balloon-like implant;
an opening in the wall at a distal end of the distal region that allows for the passage of a portion of the second catheter out of the central void or interior volume of the balloon-like implant;
the first catheter which, along with the second catheter, defines a first lumen to allow for passage of fluid from a proximal end of the first catheter to a distal end of the first catheter, and into the central void or interior volume of the balloon-like implant; the first catheter comprising:
a proximal end that is coupled to a first proximal hub; and
a distal portion that is operably coupled or joined to the opening in the wall of the proximal region of the balloon-like implant, the distal portion including a port configured to accept a guidewire;
the second catheter defines a second lumen configured to accept at least one of the guidewire and an expandable device or combinations thereof, the second catheter comprising:
a proximal end that is coupled to a proximal hub;
a proximal portion that passes through the proximal hub of the first catheter;
a distal portion that passes through the proximal opening of the balloon-like implant;
a distal portion that passes through the central void or interior volume of the balloon-like implant;
a distal portion that engages or passes through the distal opening in the balloon-like implant;
a distal end that is open;
the expandable device anchoring and supporting the balloon-like implant after being deployed, the expandable device comprising:
a first portion located at the distal end of expandable device, the first portion configured to anchor the balloon-like implant when fully deployed;
a second portion located at the proximal end of the expandable device, the second portion configured to expand within the balloon-like implant when fully deployed thereby providing support to the balloon-like implant;
a waist located between the first portion and second portion, the waist configured to engage with the opening in the wall of the distal region when fully deployed; and
wherein:
the second catheter can be moved forward or backward while the balloon-like implant remains fixed in position;
all, or a portion of, the expandable device can be placed through the lumen of the second catheter into a biological space wherein the first portion of the expandable device is adjacent to the balloon-like implant, passing through the second catheter until the waist is exposed into the biological space;
after the waist is exposed, the balloon-like implant or the first portion of the expandable device is positioned with the first or second catheter, respectively, until the waist of the expandable device is adjacent yet distal to the distal opening of the balloon-like implant and the second catheter is adjacent yet distal to the distal opening of the balloon-like implant;
the second catheter can be pulled back proximally until a distal tip of the second catheter is located in the central void or interior volume of the balloon-like implant, and a second portion of the expandable device can be deployed through the lumen of the second catheter and placed into the central void or interior volume of the balloon-like implant;
the passage of fluid through the first catheter into the central void or interior volume of the balloon-like implant can result in expansion of the balloon-like implant; and
after expansion of the balloon-like implant and placement of the second portion of the expandable device, the second catheter can be withdrawn proximally and the first catheter can be separated from the expanded balloon-like implant and removed from the patient while the balloon-like implant and the first and second portions of the expandable device remain in the patient.

2. The medical device of claim 1, wherein the balloon-like implant is a permanent implant and is configured to receive an expandable device within and the wall of the balloon-like implant comprises a flexible non-metal polymer material.

3. The medical device of claim 2, wherein flexible non-metal polymer material comprises a single layer of a non-compliant polymer.

4. The medical device of claim 1, wherein the flexible non-metal polymer material is non-compliant comprising at least one of polyamide (nylon) or polyether block amide (Pebax).

5. The medical device of claim 1, wherein the expandable device is an open cell laser cut structure or any rigid material that can be collapsed and then expanded.

6. The medical device of claim 5, wherein the open cell laser cut structure comprises nitinol.

7. The medical device of claim 1, wherein at least a portion of the wall of the balloon-like implant comprises two or more polymer layers.

8. The medical device of claim 1, wherein the balloon-like implant possesses sufficient strength to maintain itself in an expanded or partially expanded configuration in vivo after separation from the first and second catheters.

9. The medical device of claim 1, wherein the opening in the wall at the proximal region of the balloon-like implant is a proximal neck.

10. The medical device of claim 9, the proximal neck further comprising:
a buffer region having a buffer region diameter and a tapered proximal end configured to receive at least a portion of the first catheter and the second catheter; and
wherein the buffer region diameter is larger than a diameter of the proximal neck of the opening in the wall of the balloon-like implant at the proximal region, at least a portion of the buffer region configured to allow the expandable device to expand thereby preventing a push back force from prematurely or unintentionally disengaging the balloon-like implant from the first catheter.

11. The medical device of claim 10, wherein the buffer region allows for movement of the distal tip of the second catheter within the buffer region thereby moderating movement of the distal tip proximal to a latch mechanism of the first catheter when the expandable device is expanded.

12. The medical device of claim 1, wherein the adjacent first portion of the expandable device anchors the balloon-like implant by contacting a portion of the biological space outside of the balloon-like implant when deployed thereby bracing the first portion of the expandable device against the biological space.

13. The medical device of claim 1, wherein the second portion of the expandable device is positioned inside of the balloon-like implant and expanded to support the balloon-like implant by providing a rigid support structure that contacts at least a portion of the interior surface of the wall of at least the intermediate region of the balloon-like implant when deployed.

14. The medical device of claim 13, wherein by providing the rigid support structure the expandable device maximizes compression with respect to the rigid support structure providing an increased localized radial force when contacting at least the portion of the interior surface of the wall.

15. The medical device of claim 1, wherein the second portion of the expandable device is positioned inside of the balloon-like implant and expanded to support the balloon-like implant by providing a rigid support structure that includes a distal front stop located on the first portion and a back stop located on the second portion, the front and back stop resist lateral movement by engaging a portion of distal region of the balloon-like implant allowing for the expandable device to be positioned such that a known amount of expandable device material is in the biological space and in the balloon-like implant.

16. The medical device of claim 1, wherein a third catheter is engaged to the first catheter and includes a distal opening configured to accept a guidewire to pass through the opening to an area outside of the first and third catheter within the biological space.

17. The medical device of claim 16, wherein the guidewire tracks the balloon-like implant and the expandable device through a portion of the biological space, and the balloon-like implant and the expandable device are positioned within the biological space at the same time.

18. The medical device of claim 16, wherein the guidewire tracks the balloon-like implant and the expandable device through a portion of the biological space, and the balloon-like implant and the first portion of the expandable device are positioned at the same time.

19. The medical device of claim 1, wherein the expandable device is an expandable braid device.

20. The medical device of claim 19, wherein the expandable braid device includes wires in the first portion, second portion, and the waist, the first portion of the expandable braid device having a plurality of first portion regions, the second portion of the expandable braid device having a plurality of second portion regions, the braid waist located between the first portion and second portion, the waist connecting the first and second portions of the expandable braid device.

21. The medical device of claim 20, wherein at least one of the plurality of first portion regions has a first diameter and at least one other first portion region has a second diameter when deployed.

22. The medical device of claim 20, wherein at least one of the plurality of second portion regions has a first diameter and at least one other second portion region has a second diameter when deployed.

23. The medical device of claim 20, wherein the wires of at least one of the plurality of first portion regions has a first pitch and the wires of at least one other first portion region has a second pitch when deployed.

24. £ The medical device of claim 20, wherein the wires of at least one of the plurality of second portion regions has a first pitch and the wires of at least one other second portion region has a second pitch when deployed.

25. The medical device of claim 20, wherein the waist includes a dense braid of twisted wires defining a waist lumen.

26. The medical device of claim 25, wherein at least one wire runs through the waist lumen of the expandable braid device to reduce blood flow.

27. The medical device of claim 20, wherein the waist is used in part to position the second portion of the expandable braid device within the balloon-like implant.

28. The medical device of claim 25, wherein the waist is surrounded by a tube, the tube used in part to compress the dense braid of wires and to position the second portion of the expandable braid device within the balloon-like implant.

29. The medical device of claim 28, wherein the tube surrounding the waist is a marker tube, the marker tube visible when viewed with an imaging device.

30. The medical device of claim 28, wherein the tube surrounding the waist is configured to engage with the opening in the wall of the distal region of the balloon-like implant when fully deployed.

31. The medical device of claim 1 further comprising a valve mating feature, wherein the balloon-like implant includes a valve for interacting with the waist by securing a waist of the expandable device to the balloon-like implant, allowing for the second portion of the expandable device to be positioned inside the balloon-like implant without shifting the first portion of the expandable device.

32. The medical device of claim 1, wherein the expandable device includes a radiopaque material visible when viewed with an imaging device.

33. The medical device of claim 32, wherein the expandable device includes at least one single wire marker tube, a radiopaque coupler, and at least one drawn filled tube (DFT) wire with a radiopaque core.

34. The medical device of claim 20, wherein the plurality of second portion regions has a conical shape tapering from a larger distal diameter to a smaller proximal diameter.

35. An expandable braid device configured for permanent implantation in a biological space, the expandable braid device comprising:
a distal portion having a first distal diameter, a proximal portion having a first proximal diameter and generally opposite the distal portion, a waist transitioning from the distal portion to the proximal portion, a first axis extending proximal-distal between the proximal portion and distal portion, and a second axis perpendicular to the first axis;
an attachment coupler located at a proximal end of the proximal portion, the attachment coupler configured to releasably engage the expandable braid device to a delivery structure; and
a plurality of wires extending generally continuously from the proximal portion to the distal portion, the plurality of wires including a first pitch region having a first pitch.

36. The expandable braid device of claim 35 further comprising a second proximal portion diameter, and a second distal portion diameter, wherein the second portion diameters are larger than the first portion diameters.

37. The expandable braid device of claim 36 wherein the first and second diameters of the proximal portion and the first and second diameters of the distal portion are equal.

38. The expandable braid device of claim 35 further comprising a second pitch region having a second pitch, wherein the second pitch is tighter than the first pitch.

39. The expandable braid device of claim 35, wherein the plurality of wires includes a distal loop, the distal loop extending distally away from the distal portion of the expandable braid device and configured to lead the expandable braid device over a guide path whereby a guidewire engages with the distal loop and tracks along the guide path to deploy the expandable braid device.

40. The expandable braid device of claim 39, wherein the guidewire is a microcatheter or a tube having a tube lumen.

41. The expandable braid device of claim 39, wherein the distal loop includes a flared tip, the flared tip extending distal and away from the first axis.

42. The expandable braid device of claim 39, wherein the distal loop is angled between 10 and 30 degrees with respect to the first axis.

43. The expandable braid device of claim 35, wherein the waist includes a dense braid of twisted wires defining a waist lumen.

44. The expandable braid device of claim 43, wherein the waist lumen has an inner diameter of 0 mm to 0.7 mm.

45. The expandable braid device of claim 35, further comprising radiopaque materials, wherein the radiopaque materials are visible on an imaging device.

46. The expandable braid device of claim 39, wherein the expandable braid device interacts with a balloon-like implant, the balloon-like implant comprising:
a distal region, a proximal region generally opposite the distal region, and an intermediate region transitioning from the distal region to the proximal region;
a wall extending generally continuously from the proximal region through the intermediate region, to the distal region, with an exterior surface and an interior surface, the interior surface defining a central void or interior volume;
an opening in the wall at a proximal end of the proximal region that allows for the passage of fluid from a first catheter into the central void or interior volume of the balloon-like implant and also allows for passage of a portion of a second catheter into the central void or interior volume of the balloon-like implant;
an opening in the wall at a distal end of the distal region that allows for the passage of a portion of the second catheter out of the central void or interior volume of the balloon-like implant;
wherein the expandable braid device passes through the opening in the wall at the proximal region, the distal portion passing though the central void to an area outside of the balloon-like implant and the proximal portion remaining in the central void of the balloon-like implant;
wherein the proximal portion supports the balloon-like implant by contacting the interior surface of the wall in at least the intermediate region; and,
wherein the distal portion anchors the balloon-like implant and proximal portion in a desired position in biological space.

47. The expandable braid device of claim 46, wherein the waist is configured to engage with the opening in the wall of the distal region of the balloon-like implant when fully deployed.

48. The expandable braid device of claim 39, wherein a second catheter passes through a lumen of a first catheter, the first catheter comprising:
an opening located at a distal end of the first catheter;
the second catheter, wherein the expandable braid device passes through a lumen of the second catheter and the opening located at the distal end of the first catheter; and
a balloon-like implant, wherein the balloon-like implant is advanced by the first catheter through a delivery catheter.

49. The expandable braid device of claim 48, wherein the delivery catheter further comprises a third catheter engaged to and adjacent to the delivery catheter, the third catheter comprising a distal third catheter opening, wherein the third catheter is configured to receive the guidewire; and
wherein the expandable braid device and the balloon-like implant are guided over the guide path to a deployable position by the guidewire.

50. A medical device placed in a biological space, the medical device comprising:
an expanded support device having proximal and distal portions;
a balloon-like implant having a wall in contact with at least a portion of the expanded support device; and
wherein the expanded support device and the balloon-like implant are configured to resist the flow of fluid within the biological space.

51. The medical device of claim 50, wherein the balloon-like implant is a pair of opposed caps, the pair of opposed caps sealing the proximal and distal portions of the expanded support device, respectively.

52. The medical device of claim 50, wherein the expanded support device further comprises a plurality of wires extending generally continuously from the proximal portion to the distal portion, the plurality of wires including a first pitch region having a first pitch and a first diameter.

53. The medical device of claim 50, wherein the expanded support device further comprises an attachment coupler mechanism located at a proximal end of the proximal portion, the attachment coupler mechanism configured to releasably engage the expanded support device to a catheter during deployment.

54. The medical device of claim 50, wherein at least a portion of the expanded support device supports the balloon-like implant from collapsing by contacting at least a portion of the wall, wherein the wall extends generally continuously from the proximal region through the intermediate region, to the distal region.

55. The medical device of claim 50, wherein the second portion of the expanded support device supports the balloon-like implant device by providing a scaffolding structure protecting the expanded balloon-like implant from being compressed.

56. The medical device of claim 51, wherein the wall and the caps are comprised of a non-porous material.

57. A method for deploying a medical device within a biological space of a patient, the medical device comprising a collapsible balloon-like implant and an expandable device, the method comprising the steps of:
pushing the collapsible balloon-like implant through a lumen of a delivery catheter, the collapsible balloon further comprising:
a distal region, a proximal region generally opposite the distal region, an intermediate region transitioning from the distal region to the proximal region, a first axis extending proximal-distal between the proximal region and distal region, and a second axis perpendicular to the first axis;
a wall extending generally continuously from the proximal region through the intermediate region, to the distal region, with an exterior surface and an interior surface, the interior surface defining a central void or interior volume, wherein the central void or interior volume is configured to be modified based on deployment;
an opening in the wall at a proximal end of the proximal region that allows for the passage of fluid from a first catheter into the central void or interior volume of the balloon-like implant and also allows for passage of a portion of a second catheter into the central void or interior volume of the balloon-like implant;
an opening in the wall at a distal end of the distal region that allows for the passage of a portion of the second catheter out of the central void or interior volume of the balloon-like implant;
the first catheter which, along with the second catheter, defines a first lumen to allow for passage of fluid from a proximal end of the first catheter to a distal end of the first catheter, and into the central void or interior volume of the balloon-like implant; the first catheter comprising:
a proximal end that is coupled to a first proximal hub; and
a distal portion that is operably coupled or joined to the opening in the wall of the proximal region of the balloon-like implant, the distal portion including a port configured to accept a wire;
the second catheter defines a second lumen configured to accept at least one of the guidewire or the expandable device or combinations thereof, the second catheter comprising:
a proximal end that is coupled to a proximal hub;
a proximal portion that passes through the proximal hub of the first catheter;
a distal portion that passes through the proximal opening of the balloon-like implant;
a distal portion that passes through the central void or interior volume of the balloon-like implant;
a distal portion that engages or passes through the distal opening in the balloon-like implant;
a distal end that is open;
pushing the second catheter through the lumen of the first catheter into and then out of the openings in the wall of the balloon-like implant, wherein the distal tip of the second catheter is pushed distal to the opening in the wall at a distal end of the distal region of the balloon-like implant;
pushing the expandable braid device through the lumen of the second catheter and out of the distal end opening in the second catheter into the biological space exposing a flared tip of the distal portion of the expandable device. pushing a guidewire through a third catheter, the third catheter engaged and adjacent to the first catheter, wherein the guide is pushed out of an opening at the distal end of the third catheter, wherein a distal portion of the guide is exposed to the biological space after being pushed out of the opening at the distal end of the third catheter;
manipulating the guidewire while in the biological space to interact with the flared tip of the expandable device, the expandable device configured to anchor and support the balloon-like implant, the expandable device comprising:
a first portion located at the distal end of the expandable device, the first portion including the flared tip and configured to anchor the balloon-like implant when fully deployed;
a second portion located at the proximal end of the expandable device, the second portion configured to expand within the balloon-like implant when fully deployed thereby providing support to the balloon-like implant;
a waist located between the first portion and second portion, the waist configured to engage with the opening in the wall of the distal region of the balloon-like implant when fully deployed;
retracting the guidewire from the flared tip of the first portion of the expandable device back through the opening in the third catheter and into the third catheter;
expanding the first portion of the expandable device in the biological space by pushing the first portion through the second catheter until the waist is exposed in the biological space;
positioning the first portion of the expandable device within the biological space by manipulating the first or second catheters until the waist is adjacent yet distal to the distal opening of the balloon-like implant;
injecting the fluid through the lumen of the first catheter into the central void or interior volume of the balloon-like implant;
retracting the second catheter into the central void or interior volume of the balloon-like implant during fluid injection so that the waist engages with the opening in the wall of the distal region of the balloon-like implant thereby restricting fluid from flowing into and out of the central void or interior volume of the balloon-like implant;
pushing the second portion of the expandable device out of the second catheter during retraction so the second portion of the expandable device expands within the central void or interior volume of the balloon-like implant;
retracting the second catheter from the central void or internal volume of the balloon-like implant;
disengaging the first catheter from the balloon-like implant, wherein the first catheter is configured to disengage with a latch mechanism located at the proximal end of the balloon-like implant; and
removing the first and second catheters from the patient while the balloon-like implant and the first and second portions of the expandable device remain in the patient.

58. A kit for occluding a biological space, the kit comprising:
a collapsed balloon-like implant, the balloon-like implant configured to be permanently placed and expanded in the biological space;
an expandable device, the expandable device configured to anchor and support the balloon-like implant in the biological space;
a first, second, and third catheter, wherein the first, second, and third catheters are configured to deploy the balloon-like implant and the expandable device in the biological space, respectively;
a guide, wherein the guide is configured to position at least one of the balloon-like implant and the expandable device in the biological space;
a guidewire, wherein the guidewire is operably interconnected with at least one of the balloon-like implant and expandable device; and
an instruction set, wherein the instruction set is configured to instruct a user on how to use the components of the system to occlude at least a portion of the biological space.
